(19) United States
(12) Patent Application Publication
(10) Pub. No.: US 2024/0358884 A1
(43) Pub. Date: Oct. 31, 2024

(54) FERROCENE-CONTAINING POLYELECTROLYTE COMPLEX AND WOUND DRESSING

(71) Applicant: Youngstown State University, Youngstown, OH (US)

(72) Inventor: Byung-Wook Park, Youngstown, OH (US)

(21) Appl. No.: 18/140,894

(22) Filed: Apr. 28, 2023

(51) Int. Cl. A61L 15/44

(52) U.S. Cl. CPC A61L 15/44; A61L 2300/102; A61L 2300/80

(57) ABSTRACT

A composition includes a ferrocene-containing polyelectrolyte complex. Ferrocene in the polyelectrolyte complex may be bound to the polycation or the polyanion prior to combining the polycation and the polyanion. The polycation may be chitosan. The polyanion may be alginate. A wound dressing utilizes the composition.

### BACKGROUND

Chronic wounds impact millions of people each year and impose huge financial burdens on individuals and governments. In the United States alone, the treatment of chronic wounds costs the health care system $20-25 billion/year. Wound dressings have been used to clean, cover, and protect the wound sites from the external environment, and facilitate the wound healing process. Conventional wound dressings, such as hydrogel dressings have been fabricated to maintain a moist wound healing environment and conceal the wound. Some of these dressings provide a passive release of a drug to facilitate the healing process, which would be inadequate to complete the healing process. In contrast, smart wound dressings are devices that monitor and react to the wound condition by having built-in smart materials, such as stimuli-responsive materials, that can provide the therapeutic drug on-demand. These smart wound dressings have been created to effectively facilitate the wound healing process. Hydrogels may be useful as drug carriers due to their unique properties, such as high water content, biocompatibility, and biodegradability. Hydrogels are three-dimensional networks of hydrophilic polymers that can swell in water and hold a large amount of water while maintaining their structures due to cross-linking of polymer chains. Through chemical crosslinking, hydrogel polymeric chains are inter-connected by a covalent bond. In physical crosslinking, hydrogel polymeric chains interact with each other physically through secondary interactions such as ionic bonds. Polyelectrolyte complex (polyelectrolyte complex) hydrogels are classified as physically crosslinked hydrogels. They are formed by the electrostatic interactions between oppositely charged polymer chains. In general, polyelectrolyte complex hydrogels can be considered as smart materials; they can be responsive to various external stimuli, such as electric field, temperature, light, and/or pH. By applying a stimulus, polyelectrolyte complex hydrogels may offer excellent properties such as on-demand drug release, which make them attractive materials for drug delivery systems.

Topical administration of therapeutics in a controlled manner may improve the wound healing process. Precise control over the release of therapeutics from wound dressings, such as quantity and timing, are highly desirable in order to optimize wound treatment.

### BRIEF DESCRIPTION

Disclosed, in some embodiments, is a ferrocene-containing polyelectrolyte complex.

The ferrocene-containing polyelectrolyte complex may include ferrocene-conjugated chitosan as a polycation and alginate as a polyanion.

In some embodiments, the ferrocene-containing polyelectrolyte complex is formed by a process including: bonding ferrocene to chitosan to form ferrocene-conjugated chitosan; and reacting the ferrocene-conjugated chitosan with alginate.

Disclosed, in other embodiments, is a composition including the ferrocene-containing polyelectrolyte complex.

The composition may further include an active ingredient loaded in the ferrocene-containing polyelectrolyte complex.

In some embodiments, the ferrocene is bonded to a polycation of the polyelectrolyte complex.

The polycation may be chitosan.

In some embodiments, a polyanion of the ferrocene-containing polyelectrolyte complex is alginate.

A weight ratio of the ferrocene-conjugated chitosan to alginate may be about 0:1, 1:9, 2:8, 3:7, 4:6, 5:5, 6:4, 7:3, 8:2, 9:1 or 10:0, and all ranges using any two of the aforementioned ratios as endpoints. In some embodiments, the ratio is in a range of about 4:6 to about 6:4, including about 5:5 and about 5.5:4.5.

A volume ratio of the ferrocene-conjugated chitosan to alginate may be about 0:1, 1:9, 2:8, 3:7, 4:6, 5:5, 6:4, 7:3, 8:2, 9:1 or 10:0, and all ranges using any two of the aforementioned ratios as endpoints. In some embodiments, the ratio is in a range of about 4:6 to about 6:4, including about 5:5 and about 5.5:4.5.

A weight ratio of the ferrocene-conjugated chitosan to alginate may be in a range of about 1:5 to about 1:1, including from about 1:4 to about 4:9, or about 1:3.

A volume ratio of the ferrocene-conjugated chitosan to alginate may be in a range of about 1:5 to about 1:1, including from about 1:4 to about 4:9, or about 1:3.

In some embodiments, a weight ratio of ferrocene to chitosan in the ferrocene-conjugated chitosan is in a range of about 0.013 to about 0.052.

Disclosed, in further embodiments, is a wound dressing including: a cathode; an anode; and a composition located between the cathode and the anode, the composition containing: a ferrocene-containing polyelectrolyte complex; and an active ingredient.

The wound dressing may further include a polydimethylsiloxane chamber, wherein the composition is located within the polydimethylsiloxane chamber.

In some embodiments, the cathode and the anode contain laser-induced graphene (LIG).

The wound dressing may further include an adhesive layer on a side of the anode opposite the composition.

In some embodiments, a degree of ferrocene conjugation of the ferrocene-conjugated chitosan is in a range of about 55% to about 84%.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is cross-sectional view of a wound dressing in accordance with some embodiments of the present disclosure.

FIG. 2 is a partial exploded view of the wound dressing of FIG. 1.

FIG. 3 illustrates an upper electrode assembly of the wound dressing of FIG. 1.

FIG. 4 illustrates a lower electrode assembly of the wound dressing of FIG. 1.

FIG. 5 is a photograph of an experimental setup for in vitro drug release as described in the Examples section.

### DETAILED DESCRIPTION

The present disclosure may be understood more readily by reference to the following detailed description of desired embodiments included therein and the appendices which are part of the application. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure relates to a composition, a method, and a device for the controlled release of therapeutic ingredients. The composition includes a ferrocene-containing polyelectrolyte complex hydrogel. The method utilizes the hydrogel as wound dressing. The polyelectrolyte complex hydrogel may be formed from ferrocene-conjugated chitosan (polycation, positively charged) and alginate (polyanion, negatively charged). The ferrocene-conjugated chitosan may be synthesized through reductive alkylation of chitosan with ferrocene carboxaldehyde. Then, the ferrocene-conjugated chitosan may be electrostatically interacted with alginate to form the hydrogel. A ferrocene-conjugated chitosan/alginate polyelectrolyte complex has been characterized by SEM, EDX, ATR-FTIR, viscometer, turbidity meter, and fluorescence microscopy. The stability and formation of the hydrogel vary by different concentration, mixing ratio, and degree of the conjugated ferrocene. The hydrogel can be used in combination with a therapeutic active ingredient (e.g., FITC, FITC-Dextran, FITC-BSA, or vancomycin) and be placed between two electrodes (Cathode (−) and Anode (+)). Upon application of an electrical field at relatively lower potential (e.g., 1 to 3 V), the ferrocene moieties of the PEC hydrogel close to the anode are oxidized (positively charged), which provides a hydrophilic environment. On the other hand, the ferrocene moieties of the hydrogel near the cathode maintain the reduced state, which is hydrophobic. Therefore, increased polarity and water solubility of the oxidized form of the ferrocene on the anode side along with the hydrophobic nature of the reduced form on the cathode side lead to electroosmotic movement of water molecules along with the active ingredient. The magnitude of the response depends on the magnitude of the applied voltage and the degree of the conjugated ferrocene.

A hydrogel is a three-dimensional network of hydrophilic (bio)polymers that can hold a large amount of water and soluble therapeutics while maintaining its structure due to chemical or physical cross-linking of individual polymer chains. Polyelectrolyte complex hydrogels are classified as physically crosslinked hydrogels. They are formed through the electrostatic interactions between oppositely charged (bio)polymers, called polyanions and polycations. In general, polyelectrolyte hydrogels can be considered as smart materials since they can be responsive to various external stimuli, such as pH, temperature, light, magnetic field, and electric field. Polyelectrolyte hydrogels may offer excellent properties such as on-demand drug release, which make them attractive biomaterials (e.g., for use in wound dressings).

Chitosan [β-(1,4)-2-amino-2-deoxy-D-glucan], a cationic polyelectrolyte, is the N-deacetylated derivative of chitin, which is abundant in nature. Chitosan exhibits good biocompatibility, biodegradability, low toxicity, and the ability to be fabricated into various forms in tissue engineering, such as films, porous scaffolds, and hydrogels and tubes. Chitosan can be extracted from the outer skeleton of shellfish, including crab, lobster, and shrimp. Alginate, an anionic polyelectrolyte, is generally extracted from the cell walls of brown algae and is composed of sequences of α-L-guluronic acid and β-D-mannuronic acid. Alginate is biocompatible, hydrophilic and biodegradable under normal physiological conditions. Alginate has a carboxylate functional group so it can be used to form polyelectrolyte complex hydrogel.

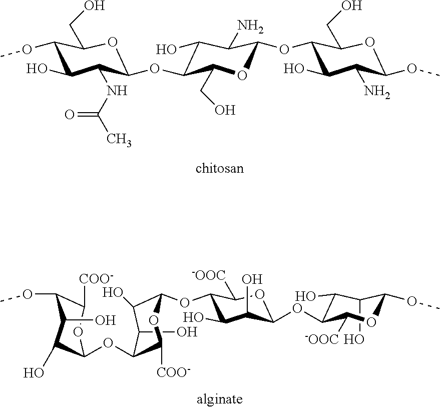

In order to form a natural electro-responsive polyelectrolyte complex hydrogel that combines the biocompatible properties of chitosan and alginate, high electron transfer efficiency is required. However, the electron transfer efficiency of chitosan and alginate is relatively low. It was unexpectedly found that this limitation can be overcome by bonding ferrocene with the polyelectrolyte.

Ferrocene is an organometallic redox (reduction/oxidation) couple mediator, which undergoes one-electron oxidation at a certain potential. Thus, it can be reversibly switched between ferrocene (Fc) and ferrocenium (Fc+). Ferrocene has attractive properties such as a reversible redox activity, rapid electron transfer, and stability in oxidized form at low potential. Ferrocene may be employed as a redox-responsive building unit of polymer due to its unique property of electrochemical redox reversibility, which also involves a reversible change between hydrophobicity and hydrophilicity.

Ferrocene-conjugated chitosan may be produced through formation of a Schiff base formed by the reaction between the aldehyde group of ferrocene and the amine group of chitosan, followed by reduction of the Schiff base.

The degree of ferrocene conjugation in chitosan chains may be determined by the ninhydrin assay. Pure chitosan was used as the fresh amine and the degree of ferrocene conjugation was calculated on the reference of fresh amine. The degree of ferrocene conjugation was found to be 58.7±3.5%, 67.8±4.4%, and 77.9±3.6%, for low ferrocene-conjugated chitosan, medium ferrocene-conjugated chitosan, and high ferrocene-conjugated chitosan, respectively. As the degree of ferrocene conjugation increases, the amount of free amine group decreases. Thus, the drug release profile under electrochemical stimulus can be further regulated by the amount of ferrocene conjugation. Then, the electrochemically active polyelectrolyte hydrogel was prepared by the electrostatic interaction of polycationic ferrocene-conjugated chitosan and polyanionic alginate. The stoichiometric ratio of polycations and polyanions in the hydrogel solutions can be determined by the turbidity measurement. The turbidity measurement can be conducted with different ratios of polycationic and polyanionic polyelectrolytes. The turbidity is expected to increase up to a certain point and then decrease, with the peak point indicating the stoichiometric ratio of the polyelectrolytes, due to the visible aggregation of polyelectrolyte complexes. The stoichiometric ratios for chitosan alginate polyelectrolyte complex, low ferrocene-conjugated chitosan alginate polyelectrolyte complex, medium ferrocene-conjugated chitosan alginate polyelectrolyte complex, and high ferrocene-conjugated chitosan alginate polyelectrolyte complex hydrogels were found to be 40%, 50%, 55%, and 55%, respectively. The shift of the stoichiometric ratio can be attributed to the point at which the positively charged functional groups of the cationic polyelectrolyte and negative charged groups are balanced. Upon the conjugation of ferrocene into the chitosan, the peak shift was found to be from 40% to 55%. It is likely that the reduced amount of the amine groups in ferrocene-conjugated chitosan results in the increase in the ferrocene-conjugated chitosan to be balanced with the polyanions (alginate). The portion of the amine groups replaced with ferrocene groups so that a larger amount of ferrocene-conjugated chitosan would be required to neutralize the anionic charges, as the charge density of the anions increases. From the FTIR analysis of the chitosan/alginate polyelectrolyte complex and ferrocene-conjugated chitosan/alginate polyelectrolyte complex hydrogels, the characteristic peaks of chitosan are obtained at 1638 and 1541 cm⁻¹. The peaks of alginate are 1591 and 1417 cm⁻¹. The peaks of chitosan/alginate polyelectrolyte complex hydrogel are seen at 1587 and 1414 cm⁻¹. The absorbance peak of chitosan at 1638 cm⁻¹ is shifted to 1655 cm⁻¹ for ferrocene-conjugated chitosan, which may be due to a reduction in primary amine N—H bending. The reduction of peak absorbance in ferrocene-conjugated chitosan/alginate polyelectrolyte complex than in chitosan/alginate polyelectrolyte complex is due to the reduction of the degree of ionic interaction between the negatively charged carboxylic ion group of alginate and the positively charged amino group of chitosan due to the introduction of ferrocene. Scanning electron microscopy (SEM) was applied for imaging the surface morphology and structure of the hydrogel samples. Compared to pure chitosan and chitosan/alginate polyelectrolyte complex hydrogel samples, the morphology of the ferrocene-conjugated chitosan and ferrocene-conjugated chitosan/alginate shows rough and flaky, which result from the conjugated ferrocene providing more open and flexible structure. It may also affect its physical properties such as pore size and drug release kinetics.

An electrochemically-regulated wound dressing transdermal patch (or “smart bandage”) includes two electrodes (e.g., laser-induced graphene (LIG) electrodes), two membranes, and a hydrogel chamber (e.g., a polydimethylsiloxane chamber). LIG is a 3D porous carbon nanomaterial formed by the laser irradiation of a polymeric precursor to induce a photochemical and thermal conversion into graphene using direct laser writing such as a CO2 laser. The ferrocene-conjugated chitosan/alginate polyelectrolyte complex hydrogel after encapsulating therapeutic molecules is placed in the hydrogel chamber well on the bottom electrode (anode). A release membrane and adhesive liner may be placed underneath the bottom electrode. The top electrode is a cathode. A membrane (e.g., a 0.14 mm thick Whatman cellulose acetate membrane with pore size 0.2 μm (Cytiva, Marlborough, MA)) may be secured to the bottom of the cathode to release therapeutic molecules while providing mechanical support for the polyelectrolyte complex hydrogel. The adhesive layer may include a silicone adhesive (e.g., Liveo Soft Skin Adhesives MG7-9700, Dupont, Wilmington, DE) and be placed on the bottom of the anode to secure the electrochemically active transdermal patch to a skin and prevent evaporation. A release liner (e.g., Scotchpak 9755, 3M, St. Paul, MN) may be used to protect the adhesive layer until applied to a skin.

The redox property of ferrocene can create a switchable wettability from its hydrophobic nature to hydrophilic charged forms upon the application of an electrochemical stimulus. Upon the electrical stimulus, the ferrocenyl groups in the ferrocene-conjugated chitosan/alginate polyelectrolyte complex hydrogel on the anode side are protonated and become hydrophilic. The natural condition of the ferrocenyl units are in its reduced state and hydrophobic, which causes water molecules migrating towards the anodic (positive) terminal of the hydrogel. When the water molecules move, they bring the model drugs (FITC, FITC-Dextran, and FITC-BSA) to the anode.

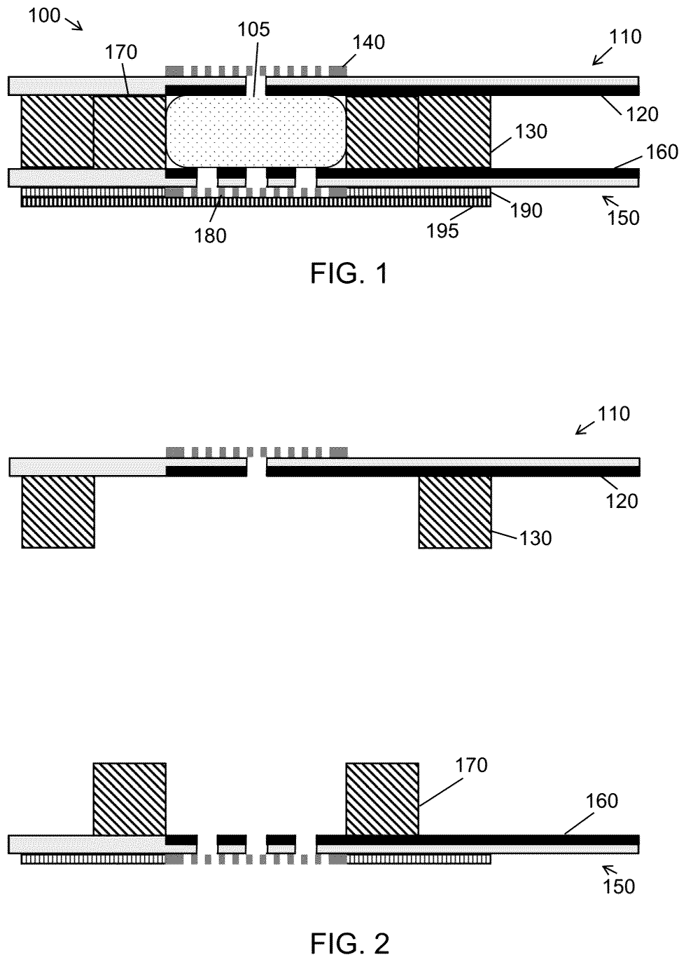

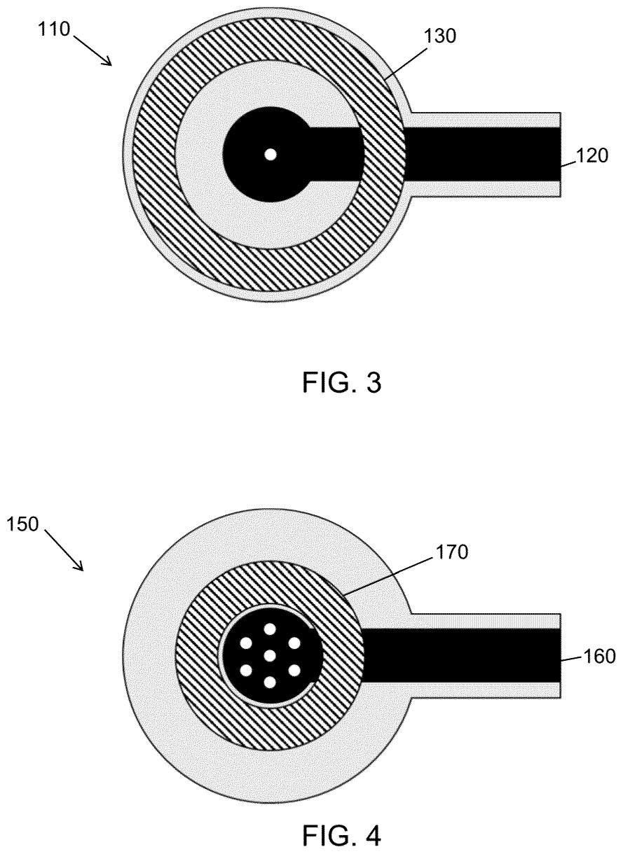

FIG. 1 is cross-sectional view of a wound dressing 100 in accordance with some embodiments of the present disclosure. FIG. 2 is a partial exploded view of the wound dressing 100 of FIG. 1. FIG. 3 illustrates an upper electrode assembly 110 of the wound dressing 100 of FIG. 1. FIG. 4 illustrates a lower electrode assembly 150 of the wound dressing 100 of FIG. 1.

The wound dressing 100 includes a polyelectrolyte complex (PEC) hydrogel 105 provided between the upper electrode assembly 110 and the lower electrode assembly 150. The would dressing 100 includes upper membrane 140, upper conductive layer (e.g., LIG conductive layer) 120, outer chamber layer (e.g., outer silicone layer) 130, inner chamber layer (e.g., inner silicone layer) 170, lower conductive layer (e.g., LIG conductive layer) 160, lower membrane 180, adhesive layer 190, and release liner 195. The adhesive layer 190 may be configured to be applied to and surround a wound to be treated after the release liner 195 is removed.

Although in the depicted embodiment the outer silicone chamber layer 130 is provided with the upper electrode assembly 110 and the inner silicone chamber layer 170 is provided with the lower electrode assembly 150, it is also contemplated that the outer layer may be provided on the lower electrode assembly and the inner layer may be provided on the upper electrode assembly. Moreover, it is possible that only one chamber layer may be included. When only one chamber layer is included, it may be provided on the upper electrode assembly or the lower electrode assembly.

Polycationic materials in this application may be any biocompatible water-soluble polycationic polyelectrolytes, for example, any polyelectrolytes or biopolymers having amine groups available as functional groups. Suitable natural and synthetic polycationic materials include chitosan, carboxymethyl chitosan, poly(L-lysine) (PLL), poly(ethylene imine) (PEI), poly(allylamine hydrochloride) (PAH), poly(D-lysine), poly(omithine), poly(arginine), poly(histidine), poly (vinylbenzyl trialkyl ammonium), poly (4-vinyl-N-alkyl-pyridimiun), poly (acryloyl-oxyalkyl-trialkyl ammonium), poly (acryamidoalkyl-trialkyl ammonium), and poly (diallydimethyl-ammonium).

Polyanionic materials in this application may be any biocompatible water-soluble polyanionic polyelectrolytes, for example, any polyelectrolytes or biopolymers having carboxylic acid groups available as functional groups. Suitable natural and synthetic polyanionic materials include alginate, hyaluronic acid (HA), pectin, dextran sulfate (DS), carrageenan, chondroitin sulfate (CS), gellan, xanthan, furcellaran, heparin, heparan sulfate, dermatan sulfate, polystyrene sulfonate (PSS), polyacrylic acid (PAA), poly(acrylic acid) (PAC), poly(vinyl sulfate) (PVS), poly(L-glutamic acid) (PGA), poly(maleic acid-co-propylene) (PMA-P), and poly(acrylamido-2-methylpropanesulfonate) (PAMPS).

Non-limiting examples of suitable reducing agents to conjugate ferrocene covalent modifier include sodium cyanoborohydride and sodium borohydride.

Where this specification refers to ferrocene, it should be understood that ferrocene derivatives as well as other metallocenes and their derivatives are also contemplated.

Non-limiting examples of active ingredients in accordance with the present application include antimicrobials, antivirals, antifungals, anti-cancer agents, anti-inflammatory agents, vitamins, minerals, analgesics, polypeptides, enzymes, RNA, DNA, metal ions, and/or nano-/microparticles.

The following examples are provided to illustrate the devices and methods of the present disclosure. The examples are merely illustrative and are not intended to limit the disclosure to the materials, conditions, or process parameters set forth therein.

### EXAMPLES

### First Set of Examples

### Materials

Low molecular weight chitosan, and alginic acid sodium salt from brown algae (medium viscosity), were purchased from Sigma-Aldrich. Ferrocene carboxaldehyde, 97%, and Sodium cyanoborohydride, 95% were obtained from Alfa Aesar. Fluorescein isothiocyanate-dextran (average Mw 3000-5000 Da) was purchased from Sigma-Aldrich. Fluorescein, pure (Mw=332 g/mol) was obtained from ACROS Organics. Agarose LE was obtained from BioExcell.

### Synthesis of Ferrocene-Conjugated Chitosan

Three different amounts of ferrocene were used in the synthesis of ferrocene-conjugated chitosan (low, medium, and high). The ferrocene amounts were adjusted in order to keep freely available amine groups to be able to interact with the alginate for polyelectrolyte complex formation. The ferrocene-conjugated chitosan was synthesized as follows. chitosan (0.9 g) was dissolved in 0.1 M acidic acid solution (360 mL deionized water, 2.058 mL acetic acid, pH=3) using a magnetic stirrer with a stirring time of 24 hours. Ferrocene carboxaldehyde (3.1, 4.7, and 9.1 mg for low, medium, and high amounts of ferrocene, respectively) was dissolved in 340 mL of methanol and then added to the first solution (while stirring), after stirring the solution (550 rpm) for 1 hour at room temperature. Sodium cyanoborohydride (1.8, 2.7, and 5.4 mg for low, medium, and high amounts of ferrocene, respectively) was dissolved in 20 ml of methanol and then added dropwise to the mixture. After 24 hours of mixing on the magnetic stirrer at 550 rpm, 23 mL of 5% sodium hydroxide was added dropwise to the mixture until precipitates formed. The products were separated by filtration and washed eight times: twice using 70% methanol, twice using 80% methanol, twice with 90% methanol, and then twice using pure methanol. The volume used for each wash was 150 mL. Finally, the products were dried overnight through evaporation, and then they were ready to be used for other experiments.

### Polyelectrolyte Complex Preparation

### Chitosan/Alginate Polyelectrolyte Complex Preparation

Alginate solution (2% w/v) was prepared by dissolving 0.4 g of alginate powder in 20 mL of deionized (DI) water. The solution was stirred for 24 hours at 500 rpm and at room temperature. chitosan solution (2% w/v) was prepared by dissolving 0.4 g of chitosan powder in 20 mL of deionized water containing 100 μL acetic acid (0.5% by weight). The solution was stirred for 24 hours. In order to form the polyelectrolyte complex, 16 mL of alginate solution was added slowly to 4 mL of chitosan solution (20% chitosan, 80% alginate) while stirring. The solution was stirred for 40 minutes at 200 rpm. The solution was centrifuged at 6000 rpm for 8 minutes to remove the captured bubbles and then poured into a petri-dish with a diameter of 3 cm. After that, the sample was placed in a HCUCFS-404 freezer (VWR, Pennsylvania, USA) at −20° C. for 24 hours then lyophilized at −50° C. in the freeze dryer to form the dried polyelectrolyte complex hydrogel.

Chitosan/alginate polyelectrolyte complex hydrogel was successfully formed through the ionic interaction between the positively charged amino group of chitosan and the negatively charged carboxylic ion group of alginate.

### Ferrocene-Conjugated Chitosan/Alginate Polyelectrolyte Complex Preparation

In order to prepare ferrocene-conjugated chitosan/alginate polyelectrolyte complex, a similar procedure to chitosan/alginate polyelectrolyte complex preparation was used. ferrocene-conjugated chitosan solution (2% w/v) was prepared by dissolving 0.4 g of ferrocene-conjugated chitosan powder in 20 mL of DI water containing 100 μL acetic acid (0.5% by weight). The solution was stirred for 24 hours. Alginate solution (2% w/v) was prepared as mentioned previously. Fifteen mL of alginate solution was added slowly to 5 mL of chitosan solution (25% chitosan, 75% alginate) while stirring. The solution was stirred for 40 minutes at 200 rpm and then centrifuged, frozen and lyophilized.

Ferrocene-conjugated chitosan/alginate polyelectrolyte complex was successfully obtained through the ionic interaction between the freely available amino groups of chitosan (that remained after the conjugation of ferrocene into chitosan, covalently through a chemical reaction) and carboxylic groups of alginate.

### Turbidity Test

The polyelectrolyte complex hydrogels were characterized by turbidity measurements, Attenuated Total Reflectance Fourier Transform Infrared Spectroscopy (ATR-FTIR), scanning electron microscopy (SEM), swelling behavior test, and gel content tests.

The turbidity measurements of the polyelectrolyte complex hydrogels were taken to find the optimum ratio between alginate, and chitosan or ferrocene-conjugated chitosan. A Thermo Scientific Orion AQ4500 turbidity meter (Thermo Scientific, Beverly, USA) was used for this purpose. The turbidity meter works by sending a light beam into the solution to be tested. The results were presented in a nephelometric turbidity unit (NTU). The concentrations 0.5, 1.0, 1.5, and 2.0% (w/v) of chitosan and alginate solutions were used to prepare different ratios of chitosan/alginate polyelectrolyte complex hydrogels. Four ratios of chitosan and alginate solutions were prepared of each concentration for this test, (20% chitosan, 80% alginate), (40% chitosan, 60% alginate), (60% chitosan, 40% alginate) and (80% chitosan, 20% alginate). All the samples were centrifuged in order to remove the captured bubbles since the bubbles affect the turbidity reading due to the change in the reflection of the light in the turbidity meter. After that, the samples were transferred to the turbidity meter's vials, and the readings were taken.

The turbidity measurements were also taken for ferrocene-conjugated chitosan/alginate polyelectrolyte complex hydrogels. 2% (w/v) solutions of alginate and the three different amounts of ferrocene-conjugated chitosan (low, medium, and high) were used to prepare different ratios of ferrocene-conjugated chitosan/alginate polyelectrolyte complex hydrogels. Turbidity tests were performed on these samples as well.

The aim of performing the turbidity test was to confirm the interaction between the chitosan or ferrocene-conjugated chitosan, and alginate, as well as to find the optimal ratio between the polyelectrolytes and the stoichiometric condition (1:1 of positively and negatively charged groups). For the chitosan/alginate polyelectrolyte complex, the highest turbidity values (NTU, Nephelometric Turbidity Unit) were noted for the ratio of (20% chitosan, 80% alginate) to be 810, 640, 515, and 320 NTU for the concentrations 2, 1.5, 1, and 0.5%, respectively for both chitosan and alginate solutions. The NTU values decreased with increasing the percentage of chitosan and decreasing the percentage of alginate in the sample.

The highest NTU values can be considered as the points of charge-to-charge balanced polyelectrolyte complex formation (1:1 stoichiometry), in which all positively charged amine groups of chitosan were ionically bonded to negatively charged carboxylic groups of alginate, that may result in the precipitation of the largest amount of polyelectrolyte complex, therefore more scattering of the turbidity meter's light.

Moreover, the NTU values increased as the concentration of both chitosan and alginate solutions increased. This can be explained by the increase in the density of the amine groups and carboxylic groups in the samples, therefore, precipitation of a larger amount of polyelectrolyte complex.

The sample at the ratio of 20% chitosan and 80% alginate and concentration of 2% (w/v) was selected as the optimized condition and characterized in terms of its mechanical stability and chemical functionality. Based on this control experiment, the ferrocene-conjugated chitosan/alginate polyelectrolyte complex hydrogel samples (including the 3 amounts of ferrocene (low, medium, and high)) were examined to find out the stoichiometric condition. The maximum turbidities at the different ratios (20% ferrocene-conjugated chitosan, 80% alginate; 25% ferrocene-conjugated chitosan, 75% alginate; 40% ferrocene-conjugated chitosan, 60% alginate) for low, medium, and high amounts of ferrocene, respectively, were found to be 870, 787, and 633.

The stoichiometric ratio between the samples that were prepared using a low amount of ferrocene was the same as the control sample (20% ferrocene-conjugated chitosan, 80% alginate), which can be explained by the availability of amine groups in ferrocene-conjugated chitosan in a number that is close to the pure chitosan, a result of conjugation of the small amount of ferrocene with free amine groups of chitosan. While the stoichiometric ratio between the samples that were prepared using a medium amount of ferrocene shifted to 25% ferrocene-conjugated chitosan, 75% alginate. A higher percentage of ferrocene-conjugated chitosan was needed at the stoichiometric ratio (25% ferrocene-conjugated chitosan) due to less availability of amine groups in ferrocene-conjugated chitosan compared to the control. The peak shift of the high amount of ferrocene was larger than in other samples, resulting from the lower amount of the free amine groups in ferrocene-conjugated chitosan.

### ATR-FTIR Analysis

The ATR-FTIR spectra of chitosan, alginate, chitosan/alginate polyelectrolyte complex, ferrocene, ferrocene-conjugated chitosan (three different concentrations of ferrocene), and ferrocene-conjugated chitosan/alginate polyelectrolyte complex were registered by a PerkinElmer Spectrum Two FTIR spectrometer with universal ATR accessory (PerkinElmer, Waltham, USA). The spectra were recorded for the dry samples between 450 and 4000 cm⁻¹ by 16 scans and it was taken 3 times for each sample, in order to detect the characteristics band of the materials, as well as to confirm polyelectrolyte complex formation.

Chitosan and ferrocene-conjugated chitosan with different amounts of ferrocene (low, medium, and high) were analyzed using ATR-FTIR. The spectra of the samples showed a decrease in the absorbance of amide I (≈1636 cm⁻¹) and amide II (≈1542 cm⁻¹) peaks with an increase in the amount of ferrocene as follows, chitosan>ferrocene-conjugated chitosan(low ferrocene)>ferrocene-conjugated chitosan(medium ferrocene)>ferrocene-conjugated chitosan(high ferrocene). This decrease in the absorbance values between the samples confirms that there is less availability of amine groups of chitosan as the amount of the reacted ferrocene increased, indicating that the ferrocene exists in the ferrocene-conjugated chitosan in a higher concentration. The decrease in the absorbance of the amine group's characteristic peaks of ferrocene-conjugated chitosan compared to pure chitosan was observed in a study that synthesized ferrocene conjugated chitosan. A new absorption band at about 814 cm⁻¹ was detected in the spectrum of ferrocene-conjugated chitosan indicating that the ferrocene has interacted with chitosan. The peak was not detected due to the reduced amount of ferrocene that was used in the reaction.

The absorbance of amide I and amide II peaks of chitosan, ferrocene-conjugated chitosan (low ferrocene), ferrocene-conjugated chitosan (medium ferrocene), and ferrocene-conjugated chitosan (high ferrocene) are summarized in the following table:

| | Amide I | | Amide II | |
|---|---|---|---|---|
| Sample | (cm⁻¹) | Absorbance | (cm⁻¹) | Absorbance |
| chitosan | 1636 | 0.075 | 1542 | 0.154 |
| low ferrocene- | 1634 | 0.041 | 1539 | 0.080 |
| conjugated | | | | |
| chitosan | | | | |
| medium | 1634 | 0.016 | 1538 | 0.033 |
| ferrocene- | | | | |
| conjugated | | | | |
| chitosan | | | | |
| High ferrocene- | 1634 | 0.008 | 1538 | 0.017 |
| conjugated | | | | |
| chitosan | | | | |

The ATR-FTIR analysis could also confirm the interaction between the polyelectrolytes by finding their characteristic peaks and observing the peak shifting. The alginate spectrum showed the characteristic band of carbonyl (C═O) at 1593 and 1406 cm⁻¹ that can be assigned to the asymmetric and symmetric stretching vibrations of carboxylate groups, respectively. Characteristic absorption bands of chitosan are usually observed between 1649 and 1652 cm⁻¹ and 1558-1598, representing amide I and amide II groups, respectively. These characteristic peaks were shifted to 1636 and 1541 cm⁻¹ due to the effect of acetic acid (used to dissolve the chitosan). The characteristic peaks of ferrocene-conjugated chitosan shifted to 1634 and 1538 cm⁻¹, which indicated that chitosan interacted with ferrocene. Disappearance of characteristic amide peaks of 1636-1541 cm⁻¹ of chitosan and 1634-1538 cm⁻¹ of ferrocene-conjugated chitosan in addition to peak shift of alginate characteristic band from 1593 cm⁻¹ to 1598 cm⁻¹ (detected in chitosan/alginate polyelectrolyte complex as well as ferrocene-conjugated chitosan/alginate polyelectrolyte complex spectra), confirms polyelectrolyte complex formation.

The intensity of the peak 1598 cm⁻¹ in chitosan/alginate polyelectrolyte complex was higher, indicating that the degree of ionic interaction between the negatively charged carboxylic group of alginate and the positively charged amino group of chitosan was stronger than that in ferrocene-conjugated chitosan/alginate polyelectrolyte complex, due to the absence of ferrocene. The characteristic peak of ferrocene was found at 1675 cm⁻¹.

### SEM Imaging and EDS

The morphologies of chitosan, ferrocene-conjugated chitosan, alginate, chitosan/alginate polyelectrolyte complex, ferrocene-conjugated chitosan/alginate polyelectrolyte complex were analyzed using (KEYSIGHT, Santa Rosa, USA) SEM with an accelerating voltage of 2 kV. Since the samples were nonconductive, they were coated with a thin layer of gold-palladium using Polaron sputter coater E5100 (Polaron, Laughton, UK) under a vacuum of 0.1 torrs and current of 20 mA, for 3 minutes. Also, energy dispersive X-ray spectrometer (EDS) analysis was carried out for chitosan and ferrocene-conjugated chitosan samples, in order to prove that the ferrocene-conjugated chitosan sample included ferrocene in the composition.

Depending on the composition, there were differences in the appearance of the fibrillar structure of the samples. The pure alginate and chitosan showed a relatively regular network due to the homogeneity of the samples. Chitosan and ferrocene-conjugated chitosan showed similar morphology.

Elemental compositions of chitosan and ferrocene-conjugated chitosan were analyzed using EDS during SEM measurements. In comparison with the chitosan, there are a few additional peaks in the spectrum of ferrocene-conjugated chitosan at 0.7 and 6.4 KeV, attributed to Fe sandwiched between two cyclopentadienyl rings in the staggered conformation of ferrocene, indicating that ferrocene was covalently conjugated with chitosan.

### Swelling Behavior

The swelling behavior of chitosan/alginate and ferrocene-conjugated chitosan/alginate polyelectrolyte complex hydrogels was determined by incubating the dry samples (size 0.5 cm³) at room temperature in phosphate buffer saline (PBS, 10 mM, pH 7.4). At first, the weight of each dry sample was measured, and then each sample was placed in 1 mL of PBS for 24 hours. After that, the wet samples were removed from the media and placed on a filter paper for 10 seconds to remove the adsorbed PBS on the surface, then weighted on the lab scale. The swelling percentage was calculated using the following equation:

$${E}_{sw} = \frac{{w}_{wet} - {w}_{dry}}{{w}_{dry}} \times 100$$

where E_{sw} is the swelling percentage, W_{wet} is the weight of the wet sample, and W_{dry} is the weight of the dry sample. For this experiment, three samples of each polyelectrolyte complex hydrogel were tested, and the average of the results was found.

The swelling ratios of chitosan/alginate, ferrocene-conjugated chitosan/alginate (low ferrocene), and ferrocene-conjugated chitosan/alginate (medium ferrocene) polyelectrolyte complex hydrogels were found to be 3480, 3692, and 4471%, respectively. The increased water uptake of the polyelectrolyte complex hydrogels with the increase of the ferrocene amount in the sample may be explained by the interaction of some of the amine groups of chitosan with ferrocene and the ferrocene-conjugated chitosan chains were able to expand more to retain a larger amount of water within the porous structure.

### Gel Content

In order to prove the stability of the chitosan/alginate and ferrocene-conjugated chitosan/alginate polyelectrolyte complex hydrogels, the gel content test was performed. Three dry samples of each polyelectrolyte complex hydrogel that had a size of 0.5 cm³ were weighed and then immersed in PBS for 24 hours. The wet samples were removed from the incubation media and then placed in the freezer (−20° C.) for 24 hours. After that, the samples were lyophilized at −50° C. in the freeze dryer. The weight of the freeze-dried samples was measured and then used in the following equation to find the gel content percentage:

$$Gel ( \% ) = \frac{{w}_{d}}{{w}_{i}} \times 100$$

where Gel (%) is the gel content percentage, W_{d} is the weight of the freeze-dried samples, and Wᵢ is the initial weight of the samples. The average of the results was found as the percentage of the gel content.

It was found that all the polyelectrolyte complex hydrogels showed almost 100% gel content, indicating that the polyelectrolyte complex hydrogels were stable in the PBS solution and confirming that the interaction between chitosan or ferrocene-conjugated chitosan, and alginate was (1:1) stoichiometric. Therefore, the hydrogels were insoluble in aqueous solution.

### Drug Release Studies

### Drug Release in Solution

In order to study the drug release behavior of chitosan/alginate and ferrocene-conjugated chitosan/alginate polyelectrolyte complex hydrogels, fluorescein-dextran (FITC-D) and fluorescein (FITC) were used as model drugs. FITC-D and FITC were dissolved in PBS at a concentration of 0.005 mg/mL. The dry polyelectrolyte complex samples were incubated in the model drug solutions for 48 hours. After that, the samples were rinsed two times each, by placing them in 1 mL of fresh PBS solution for 1 minute for each rinse. The samples were then transferred to microcentrifuge tubes containing 1 mL of PBS solution. The tubes were covered with aluminum foil, due to the sensitivity of the FITC-D and FITC to light, and then placed at room temperature. 0.5 mL of the incubation media was withdrawn and replaced with the same volume of fresh PBS solution at determined time intervals over 72 hours. During this time, the withdrawn samples were labeled and stored in the refrigerator. The released amount of FITC-D and FITC was determined using a BioTek Cytation 1 spectrophotometer (BioTek, Winooski, USA). The incubation solutions, the rinsing solutions, and the withdrawn samples were transferred to a 96-well plate (200 μl in each well) and then the intensity of the FITC-D and FITC were measured using the spectrophotometer. Fluorescence (excitation and emission wavelengths of 485 and 528 nm) and absorbance modes (absorbance at 490 nm) were used in the spectrophotometer for FITC-D and FITC solutions, respectively. In order to convert the intensity to concentration, a calibration curve was created by doing a serial dilution for FITC-D and FITC solutions that have a concentration of 0.005 mg/mL, and the intensity of the diluted solutions was measured using the spectrophotometer. The intensity values were plotted versus the concentrations and then the curve equation was found and used to convert the intensity to concentration. To calculate the amount of the incubated drug and the percentage of the released drug at a specific time, the intensity readings were converted to concentration and then the following equations were used:

$$Drug incubated in the polyelectrolyte complex hydrogel = Original concentration ( 0.005 mg / mL ) - ( The concentration of incubation solution + The concentration of rinsing solutions )$$

$$Drug released at a specific time ( e . g . , after 1 hour ) = Drug incubated in the polyelectrolyte complex hydrogel - The concentration of the solution at the time ( 1 hour )$$

$$Percentage of drug released at a specific time ( e . g . , after 1 hour ) = ( Drug released at 1 hour / Drug incubated in the polyelectrolyte complex hydrogel ) \times 100$$

The percentage of the drug released was found at each of the times of withdrawing the samples over the 72 hours and then plotted versus the time to show the drug release behavior of the samples. Three samples were used of each hydrogel for this experiment and the average was found.

The release of FITC and FITC-D from chitosan/alginate and ferrocene-conjugated chitosan/alginate polyelectrolyte complex hydrogels was investigated to evaluate their drug release behavior. In 3 days, about 70% and 83% of the FITC were released, respectively, from chitosan/alginate and ferrocene-conjugated chitosan/alginate polyelectrolyte complex hydrogels. The release rate of FITC from chitosan/alginate polyelectrolyte complex hydrogel was lower due to the less amount of FITC molecules that were encapsulated into the chitosan/alginate polyelectrolyte complex hydrogel. This result may be explained by the swelling behavior that showed a lower swelling ratio for chitosan/alginate polyelectrolyte complex hydrogel among other polyelectrolyte complex hydrogels. The drug release behavior can also be attributed to the strength of the ionic bond between the polyelectrolytes. The ATR-FTIR shows that the ionic bond was stronger in chitosan/alginate polyelectrolyte complex. Therefore, the entrapped FITC may be released at a slower rate. It is likely that the electrostatic interaction between ferrocene-conjugated chitosan and alginate in ferrocene-conjugated chitosan/alginate polyelectrolyte complex is lower due to the possible interaction of some of the amine groups on chitosan with ferrocene, resulting in a faster release rate of the loaded FITC molecules.

The release of FITC-D (a model drug with a large molecular weight of 2000-3000 Da) from the polyelectrolyte complex hydrogels was also investigated in order to test the drug release behavior of the polyelectrolyte complex hydrogels with two different model drugs with different molecular weights. About 38% and 61% of the FITC-D were released, respectively, from chitosan/alginate and ferrocene-conjugated chitosan/alginate polyelectrolyte complex hydrogels over a period of 3 days.

### Drug Release on Agarose Gel (Passive)

In order to study the drug release on agarose gel (acting for research purposes as a phantom skin), the experiment was designed as follows. A polydimethylsiloxane (PDMS) sheet was created by mixing 20 mL of elastomer base with 2 mL of curing agent (10:1 mixing ratio) for 5 minutes and then poured into a petri-dish (diameter=10 cm). The captured bubbles were removed by placing the petri-dish in a vacuum pump with the pressure gauge reading between −20 to −23 bar, for 30 minutes. Afterward, the sample was placed in the oven at 80° C. for 2 hours to obtain an elastic PDMS sheet. The thickness of the resultant PDMS sheet was about 0.5 cm. The sheet was used to prepare a mold to hold the agarose gel. The agarose gel was prepared by dissolving 0.6 g of agarose powder in 30 mL of PBS solution and then microwaved for 4 minutes in order to have a fully homogenous solution. Using a micropipette, the liquid agarose solution was poured into the PDMS mold to create an agarose gel layer, the agarose solution was allowed to leak under one side of the mold to create an attached thin layer of agarose gel that will be used as a base to the polyelectrolyte complex hydrogels, to prevent the leakage of the drug released under the layer that acts as phantom skin. The agarose solution was left for 2 minutes to cool and solidify. It was then ready for use in the experiment.

The passive release behavior of chitosan/alginate and ferrocene-conjugated chitosan/alginate polyelectrolyte complex hydrogels was studied using FITC-D and FITC as model drugs. FITC-D and FITC solutions were prepared in a concentration of 0.005 mg/mL in PBS solution. The polyelectrolyte complex samples were prepared in size of (1 cm×0.5 cm×0.5 cm) and then immersed in the model drug solutions for 48 hours. Afterward, the samples were taken from the incubation media and rinsed for 1 minute twice using fresh PBS solution. Then, the samples were placed on a filtration paper for 10 seconds to remove the excess solution on the surface. After that, each sample was carefully placed attached to the prepared agarose gel. The whole assembly was then transferred to Zeiss Axio Vert A1 inverted microscope (Zeiss, Oberkochen, Germany) in order to image the movement of the model drugs through the agarose gel. An image was taken every 10 seconds. The fluorescence mode was carried out with a FITC channel, a LED light intensity (30%), an exposure time (500 ms), and a 20× objective lens. The middle of the agarose gel layer was focused during the time-lapse imaging.

The drug release kinetics of the polyelectrolyte complex hydrogels were investigated on the surface of agarose gel to represent a similar condition to the body skin. Time-lapse images of the movement of the model drugs through agarose gel were collected (at the interface between the polyelectrolyte complex hydrogels and agarose gel) for testing chitosan/alginate and ferrocene-conjugated chitosan/alginate polyelectrolyte complex hydrogels in passive (no electrical stimulus) and active (with electrical stimulus) manners. The images clearly showed the enhanced release of the model drug with electrical stimulus, represented by the significant increase of the intensity of FITC that diffuses through the agarose gel with time. Also, these images were analyzed to find the change in the intensity profiles of the model drugs with time. Charts showing the spatiotemporal changes in the fluorescence intensity of FITC and FITC-D, respectively, within the bulk agarose gel (phantom skin) were prepared. Large deviations were observed in the collected data due to variations in the samples of the polyelectrolyte complex hydrogels from batch to batch.

With no electrical stimulus, the passive release of FITC-D through agarose gel was slower compared to FITC release, which was apparent by comparing the intensity values at the interface to be 5500 a.u. for FITC and 4200 a.u. for FITC-D at the same time (5 min), the lower intensity proves the slower release of FITC-D, and that refers to the larger molecular weight. So, the slower release rate from the polyelectrolyte complex hydrogels is expected with the FITC-D.

### Drug Release on Agarose Gel (Active)

In order to study the active release of the polyelectrolyte complex hydrogels, a voltage was applied to demonstrate the effect of the electric field on the release behavior. The same experiment design with the same conditions as explained for the passive release was used for the active release. The only difference was the addition of two platinum (Pt) wires as electrodes that were fixed on the petri dish lid (the petri dish that held the agarose gel layers inside) by creating a slit in the lid and fixing the wires (4 mm apart) using PDMS blocks. The Pt wires were connected to a power supply, the cathode electrode was inserted into the polyelectrolyte complex hydrogel sample in the front (in a contact with the phantom skin), and the anode in the back.

Two voltage values (0.25 and 1 V) were applied to stimulate the polyelectrolyte complex hydrogels, and the movement of FITC and FITC-D through the agarose gel overtime was imaged.

Upon the application of electrical stimulus, both polyelectrolyte complex hydrogels (chitosan/alginate and ferrocene-conjugated chitosan/alginate) were shown to be electrically responsive. The changes in the fluorescence intensity at the interface under the electrical stimulus were found to be larger than those in no electrical stimulus. Also, it was found that the degree of the electrical potential (0.5 or 1.5 V) directly affects the release kinetics, resulting in the changes in the fluorescence intensity. The higher electrical stimulus may lead to the enhanced electro-osmosis; therefore, a faster release rate that represented by the significant increase in the intensity values (16000 a.u at t=5 min) for the active release (1.5V), compared to the intensity values (7000 at t=5 min) for the active release (0.5 V). To compare the electro-responsivity of the polyelectrolyte complex hydrogels (chitosan/alginate and ferrocene-conjugated chitosan/alginate) in terms of electro-responsivity, the difference of the fluorescence intensities at specific incubation times for both passive and active releases were found to be higher with the ferrocene-conjugated chitosan/alginate polyelectrolyte complex hydrogel, indicating that it is more responsive to the electric field proved by the significant increase in the intensity profile. This result may be attributed to the redox mediator of ferrocene that increases the electro-osmosis and development of a stress gradient in the polyelectrolyte complex hydrogel.

The condition of the functional groups of the polyelectrolytes plays a major role in the electro-responsivity of polyelectrolyte complex hydrogels. Without wishing to be bound by theory, a proposed mechanism of the electro-responsivity of the polyelectrolyte complex hydrogels is explained as follows. When the voltage is applied, a local decrease of the pH occurs around the anode and a neutral pH around the cathode. The free amine groups available in the chitosan/alginate polyelectrolyte complex hydrogel are protonated, and the carboxyl groups available in the polyelectrolyte complex hydrogel are neutralized on the anode side due to the low pH. On the cathode side, however, the carboxyl groups are deprotonated, and the amine groups are neutralized. Thus, the protonated amine groups would be attracted to the cathode side, whereas the negatively charged carboxyl groups are attracted to the anode side, resulting in pushing the model drug out, therefore, an active release occurs.

With the ferrocene-conjugated chitosan/alginate polyelectrolyte complex hydrogel, the enhanced electro-responsivity compared to the chitosan/alginate polyelectrolyte complex hydrogel may be explained as follows. Ferrocene is an organometallic redox couple (ferrocenium/ferrocene, ferrocene+/ferrocene) mediator, which undergoes one-electron oxidation at a certain potential. Upon the electrical stimulus, the oxidized ferrocene (ferrocenium) together with the protonated amine groups of chitosan would have a higher density of the protonated functional groups in the polyelectrolyte complex hydrogel compared to the deprotonated carboxyl groups. The relatively larger amount of the positively charged functional groups on the cathode side may provide a higher electro-osmosis followed by a higher pressure gradient in the polyelectrolyte complex hydrogel. The enhanced driving force may induce the model drug molecules released more at the cathode side, compared to the case of the chitosan/alginate polyelectrolyte complex hydrogel with no ferrocene.

### Diffusion Coefficient Calculations

The diffusion coefficient of the model drugs through the agarose gel can be estimated by fitting the intensity profiles of the FITC-D and FITC that were produced during the imaging using the microscope with an appropriate model based on the solution of Fick's second law,

$$\frac{\partial c ( x , t )}{\partial t} = D \frac{{\partial }^{2} c ( x , t )}{\partial {x}^{2}}$$

where c is the concentration, x is the distance, t is the time, and D is the diffusion coefficient.

The boundary conditions in the experiment were:

$$C = 0 , t \to 0 , x > 0$$

$$C \to ∞ , t \to 0 , x = 0$$

The solution of led to the following equation:

$$\frac{c ( x , t )}{{c}_{0}} = \frac{1}{2 \sqrt{π D} t} exp ( \frac{- {x}^{2}}{4 D t} )$$

This equation was applied to experimental data in the form of:

$$I ( x , t ) = {P}_{1} exp ( - {P}_{2} {x}^{2} )$$

where I is the intensity of FITC-D and FITC in arbitrary units and P1 and P2 are the fitting parameters. From these equations:

$${P}_{2} = \frac{1}{4 D t}$$

$$D = \frac{1}{4 {P}_{2 t}}$$

In order to solve the equation and find the values of P1 and P2, a custom-built MATLAB code was written and run to find P1 and P2, followed by D.

The diffusion coefficients (D) of the passive and active releases were estimated by fitting the intensity profiles with an appropriate model based on the solution of Fick's second law. The D values obtained from different intensity profile curves at different times (for ferrocene-conjugated chitosan/alginate polyelectrolyte complex and FITC as a model drug are summarized in the table below.

| | | Time (min) | | |
|---|---|---|---|---|
| | | 1 | 3 | 5 |
| Passive, 0 V | P₁ | 4055 ± 50 | 4481 ± 137 | 5099 ± 223 |
| | P₂ × 10⁷ | 6.8 ± 1.4 | 10 ± 1.6 | 15 ± 1.4 |
| | D × 10¹⁰ (m²/s) | 63.3 | 14.3 | 5.7 |
| | SD × 10¹⁰ | 12.1 | 2.4 | 0.5 |
| Active, 0.5 V | P₁ | 4387 ± 253 | 4970 ± 277 | 6179 ± 298 |
| | P₂ × 10⁷ | 8.5 ± 3.8 | 1.2 ± 4.2 | 20.4 ± 4.17 |
| | D × 10¹⁰ (m²/s) | 60.9 | 12.2 | 4.2 |
| | SD × 10¹⁰ | 28.2 | 3.5 | 0.8 |
| Active, 1.5 V | P₁ | 5380 ± 133 | 12426 ± 678 | 13601 ± 300 |
| | P₂ × 10⁷ | 26.5 ± 144 | 65.7 ± 1.7 | 68.8 ± 1.1 |
| | D × 10¹⁰ (m²/s) | 15.8 | 2.1 | 1.2 |
| | SD × 10¹⁰ | 0.89 | 0.06 | 0.02 |

The averaged D values for the different time points were found to be 27.7±31.0, 25.7±31.0, and 6.4±8.0 m²/s for passive, active 0.5V, and active 1.5V releases. The apparent D values with the large deviations indicate there is no statistically significant difference between the averaged D values. The number of experiments needs to increase, so that the statistical analysis is carried out properly. Based on the results from the previous studies and our experimental result, it is confirmed that the estimated D values should be constant, regardless of the different amounts of the model drug released from the polyelectrolyte complex hydrogels.

### Second Set of Examples

### Materials

Sigma Aldrich was the supplier for various chemical substances used in these examples, including low molecular weight chitosan (CHI) powder derived from deacetylated chitin, medium viscosity alginate (ALG), fluorescein isothiocyanate-dextran (FITC-Dextran) with a molecular weight range of 3000-5000, and albumin fluorescein isothiocyanate conjugate protein bovine (FITC-BSA) with a molecular weight of 66 kDa. The reducing agent used in the experiment, sodium cyanoborohydride (NaBH₃CN), was also obtained from Sigma Aldrich. Ferrocene carboxaldehyde (Fc) was purchased from Alfa Aesar, while fluorescein (FITC) was obtained from ACROS organics. Ninhydrin (Monohydrate, Reagent) was purchased from spectrum chemicals. VWR supplied sodium hydroxide (NaOH) with a concentration of 50% w/w, and ninhydrin reagent was obtained from Spectrum Chemicals. Ciprofloxacin (CIP) was obtained from Enzo Life Sciences. In addition to these chemicals, Dow Chemicals provided elastomers for Polydimethylsiloxane (PDMS) preparation, and ATCC supplied both Staphylococcus aureus (S. aureus) (ATCC 29213) and Pseudomonas aeruginosa (P. aeruginosa) (ATCC 27853). Cellulose acetate filter membranes were purchased from Whatman, Sigma Aldrich, and Hardy Diagnostics offered 2 kgs lysogeny broth (LB) with combinations of tryptone 10 grams, sodium chloride 5 grams and yeast extract 5 grams for use in bacterial culture experiments. The supplier for LB agar for bacterial growth was VWR, while 500 grams 5X-M9 salt with disodium phosphate 33.9 grams, monopotassium phosphate 15 grams, sodium chloride 2.5 grams and ammonium chloride 5 grams for bacterial media preparation could be purchased from BD and BD chemicals. Agarose LE is obtained from EXCEL.

### Synthesis of (Fc-CHI) and Formation of PEC Hydrogel

To prepare the CHI solution, 1.5 weight percent (wt. %) of CHI was mixed with 0.1 M acetic acid solution. The ALG solution was prepared by mixing 1.5 wt. % of ALG with deionized water at 60° C. The CHI/ALG PEC was then prepared by mixing the CHI and ALG solutions at different volumetric ratios, such as 1:9, 2:8, and so on. For example, a 1:9 CHI/ALG solution refers to; for every 1× volume of CHI solution, there are 9× volumes of ALG solution. This process results in the synthesis of the CHI/ALG PEC at different ratios.

For the synthesis of Fc-CHI solution, CHI solution was first prepared by dissolving 0.9 grams of CHI in 360 ml of 0.1 M acetic acid solution. Three different concentrations of Fc-CHI were then prepared by adding different concentrations of Fc solution to the CHI solution. The Fc solution was prepared in 340 ml methanol with three different concentrations. 0.06 mM, 0.12 mM, and 0.18 mM Fc were used to make low, medium, and high concentration Fc solutions, respectively. The volume of the Fc solution and CHI solution were kept the same for each concentration of Fc-CHI. This process will result in the synthesis of low, medium, and high concentrations of Fc-CHI.

After mixing Fc solution with CHI solution for one hour, a reducing reagent solution containing different amounts of sodium cyanoborohydride was added to the Fc-CHI solution. The reducing reagent solution was prepared in 20 ml methanol with 0.12 mM, 0.24 mM, and 0.36 mM of sodium cyanoborohydride for the low, medium, and high Fc-CHI solutions, respectively. The resulting solution was then mixed with Fc-CHI solution on a magnetic stirrer at 500 rpm for 24 hours at room temperature. After this, the solution was titrated with NaOH (5 vol %). The orange precipitate that forms was then filtered four times with methanol solutions of increasing concentration (first with 70% methanol, second with 80% methanol, third with 90% methanol, and fourth with 100% methanol). The UV spectroscopy of the filtrate obtained at each step was used to confirm that there were no free Fc molecules present in the Fc-CHI residue. Finally, Fc-CHI granules were obtained after incubating the residue for 24 hours at 32° C.

To prepare the Fc-CHI solution, 1.5 wt. % (0.75 grams) of Fc-CHI was dissolved in 0.1 M acetic acid solution (50 ml). The ALG solution was prepared by mixing 1.5 wt. % (0.75 grams) of ALG with 50 ml of DI water at 60° C. The Fc-CHI/ALG PEC was then prepared by mixing the Fc-CHI and ALG solutions at different ratios, such as 1:9, 2:8, and so on. The resulting Fc-CHI/ALG PEC solution was then lyophilized with Freeze dryer ‘Labconco Freezedryer’, Marshall Scientific, USA. The lyophilization is performed at −53° C. and 0.123 Torr for 24 hours.

### Characterization of PEC Hydrogels

### Ninhydrin Assay

The degree of conjugation of CHI can be measured using ninhydrin. The number of amino groups present in the CHI and its conjugates can be quantified using this method. Conjugation refers to the formation of chemical bonds between polymer chains, which can increase the strength and stability of the material. The amount of free amino groups in the test sample is proportional to the optical absorbance of the solution after heating with ninhydrin. This means that the higher the absorbance, the more amino groups are present in the sample. By comparing the absorbance of the different samples, it is possible to estimate the degree of crosslinking in the Fc-CHI hydrogels.

To do this, a ninhydrin solution is made by mixing ninhydrin powder with Ethyl alcohol at a concentration of 20 mg/ml. This solution is then mixed with CHI and CHI conjugates (such as Fc-CHI with low, medium, and high concentrations of Fc) in a 1:1 ratio. The mixture is then incubated at 50° C. for 1 hour at 100 RPM. Since, ninhydrin is reactive to light, the incubation is done covering the mixture in aluminum foil. After that the mixture is cooled to the room temperature and the absorbance is read at 570 nm using Spectrophotometer (Bio TEK, USA) in 96 well plate.

The degree of conjugation is obtained by:

$$Degree of conjugation = \frac{NHN reactive {amine}_{fresh} - NHN reactive {amine}_{fixed}}{NHN reactive {amine}_{fresh}} \times 100$$

Where, NHN reactive amine_{fresh} is the absorbance of light by fresh amine i.e., pure CHI, and NHN reactive amine_{fixed} is the absorbance of light by amine mixture i.e., Fc-CHI.

### Turbidity

Turbidity is an optical property that describes the amount of light that is scattered or absorbed by the suspended particles as it passes through a liquid. The maximum turbidity obtained helps to determine the stoichiometric proportion between the components.

PEC solution of pure CHI/ALG, low Fc-CHI/ALG, medium Fc-CHI/ALG and high Fc-CHI/ALG was prepared in different volumetric ratios of pure CHI or Fc-CHI to ALG. The Fc-CHI was made with 1.5 weight %. The ALG solution was also prepared 1.5 weight %. These individual solutions were mixed in the ratio of pure CHI or Fc-CHI:ALG in 0:1, 1:9, 2:8, 3:7, 4:6, 5:5, 6:4, 7:3, 8:2, 9:1 and 10:0. The turbidity of all PEC solution with all ratios was measured with Orion AQ4500 turbidity meter (Thermo Scientific). The PEC solutions were prepared for 3 times, and its turbidity was measured each time and the average is taken.

### Viscosity

Viscosity test is performed to determine the mechanical strength of the polymer solution. The viscosity of pure CHI/ALG, as well as low, medium, and high Fc-CHI/ALG solutions, was determined at their respective stoichiometric proportions. The stoichiometric proportions were established based on the results of the turbidity test and the measurements were taken at room temperature using a rotational viscometer (Fungilab Viscolead, Barcelona, Spain). The viscosity of PEC solutions was evaluated using an L2 spindle with a range of viscosity measurements from 300 cP to 100,000 cP at various rpm. The viscosity was determined at 12 rpm.

### Fourier-Transform Infrared Spectroscopy (FTIR)

FTIR analysis was carried out using Attenuated Total Reflectance Fourier Transform Infrared Spectroscopy (ATR-FTIR) (PerkinElmer, Massachusetts, USA) with wavelength between 400-4000 cm⁻¹ and 16 scans. The peak of the absorbance obtained helps in predicting the functional groups present in the sample. All the PEC samples were lipolyzed before taking spectra. Pure CHI, pure ALG, pure Fc flakes, pure CHI/ALG with stoichiometric proportion and high Fc-CHI/ALG with stoichiometric proportion was studied.

### SEM/EDS Imaging

The hydrogel samples of pure CHI, pure ALG, pure CHI/ALG, and high Fc-CHI/ALG were imaged with a JEM-2100F scanning/transmission electron microscope (JEOL, Tokyo, Japan) using acceleration voltage of 12 keV and 15 kV beam current. SEM images can reveal the microstructure of hydrogels, including their pore size, distribution, and interconnectivity, which can be correlated with the swelling behavior and mechanical properties of the hydrogel. EDS can also identify the elemental composition of the hydrogel, which can provide insights into its chemical properties and potential interactions with biological systems. To prepare samples for SEM/EDS, the samples were mounted on aluminum stub with the help of carbon tape. Adequate height was raised, and the sample was inserted inside the chamber of SEM microscope.

### Swelling Behavior

A 5 mm³ sponge of PEC was taken, weighted, and incubated in PBS to study the swelling behavior. Rate of swelling depend on the chemical and physical properties of the hydrogel, such as its crosslinking density, porosity, and charge density. Swelling can affect the drug release behavior by controlling the diffusion of the drug molecules through the hydrogel network. The sponge of pure CHI, pure ALG and Fc-CHI dissolved in the PBS due to weak ionic bond. This ionic strength is increased by crosslinking CHI and ALG together. Swelling ratio test of the stoichiometric CHI/ALG, low Fc-CHI/ALG, medium Fc-CHI/ALG and high Fc-CHI/ALG was performed. After 24 hours of incubation, their weight was measured by drying in KimTECH tissue for 10 second. Swelling ratio was calculated as:

$$Swelling ratio = \frac{{W}_{wet} - {W}_{initial}}{{W}_{initial}} \times 100$$

W_{wet} is the weight of the PEC after being soaked in PBS. Wᵢₙᵢₜᵢₐₗ is the weight of the PEC before soaking in PBS.

### Gel Content

A 5 mm³ PEC was taken, weighted, and incubated in PBS. After incubating for 24 hours, excess PBS was removed from the surface and lipolyzed in a fridge dryer at −50° C. for another 24 hours. Gel content was measured as:

$$Gel Content = \frac{{W}_{dry}}{{W}_{initial}} \times 100$$

Where, W_{dry} is the weight of the lipolyzed PEC. Wᵢₙᵢₜᵢₐₗ is the weight of the PEC before soaking in PBS.

### Drug Release Kinetics

The release of different hydrophobic and hydrophilic drug gives the proper explanation for the overall release pattern from the specific material. The electrochemical properties and release kinetics of the PECs were evaluated using a fluorescent dye called FITC. FITC-Dextran and FITC-BSA were used as the model drugs.

The release platform was prepared with agarose LE, made by dissolving 2 weight % agarose LE in PBS solution. This mixture was boiled and kept at 60° C. for maintaining in liquid state. Once cooled to room temperature, it solidified within 2 minutes. Different PECs (pure CHI/ALG, low Fc-CHI/ALG, medium Fc-CHI/ALG and high Fc-CHI/ALG) were soaked in model drugs solution for 24 hours. FITC, FITC-Dextran and FITC-BSA solutions of 0.5 mg/ml, 1 mg/ml and 1 mg/ml concentration were selected to be the incubation solution, respectively. Before releasing FITC into agarose structure excess solution and impurities from the surface of PEC was removed by washing the incubated PEC 2 times in the pure PBS solution for 1 minute each time.

### Concentration of the Drug Uptake by Different PEC.

| FITC (0.5 mg/ml) | | FITC-Dextran (1 mg/ml) | | FITC-BSA (1 mg/ml) | |
|---|---|---|---|---|---|
| Pure CHI/ALG | 0.02 | Pure CHI/ALG | 0.052 | Pure CHI/ALG | 0.248 |
| Low Fc-CHI/ALG | 0.15 | Low Fc-CHI/ALG | 0.103 | Low Fc-CHI/ALG | 0.318 |
| Medium | 0.22 | Medium | 0.117 | Medium | 0.401 |
| Fc-CHI/ALG | | Fc-CHI/ALG | | Fc-CHI/ALG | |
| High Fc-CHI/ALG | 0.28 | High Fc-CHI/ALG | 0.158 | High Fc-CHI/ALG | 0.421 |

The concentration of FITC absorbed by the hydrogel was calculated by creating a calibration curve using solutions with varying concentrations of FITC. The absorbance intensity was measured at 495 nm. The concentrations of the incubation and wash solutions was determined using the calibration curve. Finally, the exact concentration in the PEC was found by subtracting the concentrations of the incubation and wash solutions from the original solutions. This method was similar to the one used in a study by the team of Yu Wang in 2022. The concentration of the FITC absorbed by pure CHI/ALG, low Fc-CHI/ALG, medium Fc-CHI/ALG and high Fc-CHI/ALG was 0.02, 0.15, 0.22 and 0.28, respectively. The concentration of FITC-Dextran up taken by pure CHI/ALG, low Fc-CHI/ALG, medium Fc-CHI/ALG and high Fc-CHI/ALG was 0.052, 0.103, 0.117 and 0.158 respectively. Similarly, the concentration of FITC-BSA loaded into pure CHI/ALG, low Fc-CHI/ALG, medium Fc-CHI/ALG and high Fc-CHI/ALG was found to be 0.248, 0.318, 0.410 and 0.421, respectively. The release of FITC from the PECs to the agarose structure was then observed using a fluorescent microscope and measured in terms of the intensity of the agarose material contacted to PECs. The kinetics of release was measured with two approaches i.e., static voltage application and cyclic voltage application.

### Electrical Stimulus: Continuous Mode

When it comes electrochemical cells, there are two types: galvanic and electrolytic. Galvanic cells derive its current flow from redox reactions taking place on the electrodes, while electrolytic cells need an external electrical source like a DC power supply. In order to apply the electrical stimulus onto the E-PEC hydrogel, the anode was taken to be positive while the cathode was negative. The drug was loaded into a PEC, which was prepared by soaking it in a 0.5 wt. % drug solution for 24 hours. PECs with varying concentrations of Fc were loaded with the drugs and washed twice with PBS. The drug-loaded PEC was then placed next to the agarose gel, and the passage of the drug was observed under a fluorescence microscope with specific settings. The particular configuration consists of the choice of 20× magnification lens, 15 seconds of exposure of 50% intensity FITC light, and 10 second interval between each data point. A constant electric stimulus was applied at different voltages and currents to release the drug. The time taken for the release of FITC, FITC-Dextran, and FITC-BSA were 5, 10, and 15 minutes, respectively. The initial intensity of each datapoint was recorded to create an intensity profile plot.

Electrical Stimulus: Switching Mode (on/Off)

This experiment is performed to study the release of FITC-D, and FITC-BSA from PECs from PEC hydrogels in response of electric field. The PECs are incubated in solutions containing different concentrations of the drugs (1 mg/ml FITC-D, and 1 mg/ml FITC-BSA). The PECs are then placed next to a 2 wt. % agarose structure and the diffusion of the drugs from the PECs is observed through the intensity change. Intensity was recorded using a ZEISS AxioVert A1 microscope with 20× lens, at interval of 10 s. The electric stimulus is turned on and off at every 30 s. A curve is generated from the recorded intensity values over time, and the average rate of release is calculated from the slope of the individual points on the curve. The obtained intensity profile helps to predict the responsiveness of the respective PEC with the electric field.

### In-Vitro Drug Release

This experiment was performed to study the interactions between the hydrogel and other substances, such as drugs. Once the hydrogel with maximum electric responsivity is declared, this in-vitro experiment is carried out to see the effectiveness of the PEC in releasing the drug to the biological environment.

The release was carried out on S. aureus biofilm and P. aeruginosa biofilm. Biofilm was prepared with liquid bacterial culture of 0.05 OD. 100 μl of liquid bacterial culture was dropped over the acetate filter membrane, kept over LB agar and incubated at 37° C. for 48 hours. The growth of biofilm is seen over the period of 48 hours.

Antibiotics were applied to the biofilm for different durations: 0 minutes, 30 minutes, 1 hour, 3 hours, and 6 hours, encapsulating 200 μg/ml, which was the minimum concentration required to eradicate the S. aureus. To synthesize the CIP-loaded electrochemically active polyelectrolyte hydrogel (CIP-E-PEC), Fc-CHI/ALG was incubated in a 1000 μg/ml CIP solution for 24 hours and 195 μg/ml CIP was loaded into the hydrogel. This method was followed in the reference of task performed by Demiana where drug release rate was studied with incorporation of different amount of CIP. The CIP/E-PEC was then placed over the biofilm for a fixed duration and removed. The biofilm was diluted with M-9 salts by adding it to a 15 ml Falcon tube containing 10 ml of 1× M-9 salts. The biofilm was dislodged from the filter with a glass rod using aseptic technique and vortexed for homogenization. This bacterial solution was diluted to 10⁻⁷ dilutions with 1× M-9 salt. A 100 μl bacterial solution was then plated on an LB agar plate, and the CFUs were counted after 24 hours of incubation. To compare the effectiveness of hydrogel, antibiotics, and electrical field in inhibiting bacterial growth, the activity of the bacteria was observed using E-PEC with PBS, E-PEC with electric field only (E-PEC+ Stimulus), E-PEC with CIP (CIP/E-PEC), and CIP loaded E-PEC with electrical stimulus (CIP/E-PEC+ Stimulus). Sayeed Hasan and his team also used a similar approach to inhibit S. aureus by applying Chitosan hydrogel. Here, the electric stimulus at 2V was supplied through the electric device. The hydrogel was encapsulated inside the device and was placed on top of the biofilm formed on the LB agar plate.

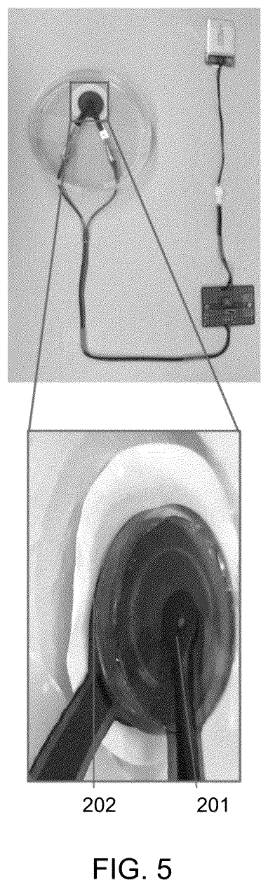

FIG. 5 is a photograph of the experimental setup, including PEC loaded with CIP encapsulated in the electronic device 201 and biofilm 202.

### Statistics

The characterization method involved ninhydrin test, turbidity test, viscosity measurement, FTIR measurement, SEM/EDS analysis, gel content and swelling ratio test. Ninhydrin test, turbidity test, viscosity measurement, gel content and swelling ratio tests were performed three times. The collected data was then used to determine the average and standard deviation using Microsoft Excel. The drug release kinetics were analyzed using the same process, taking the average of ten data points, and calculating the standard deviation. The in-vitro drug release experiment was conducted three times and the average and standard deviation were calculated from the combined data using the same method.

### Results and Discussion

The properties of the hydrogel to be used for the wound healing mainly should have polarity to help in migration of the drugs, proper porosity, structural integrity, high water retention and responsivity towards the external stimulus. Here, CHI/ALG PEC is formed by the attraction between positively charged free amines (—NH₂) of CHI and negatively charged free carboxylic group (—COOH) of ALG. This attraction results in the formation of a stoichiometric ionic bond between CHI and ALG. When Fc is added, a covalent bond is formed between Fc and CHI to produce Fc-CHI. Once the covalent bond is formed, the ionic bond is formed with the free carboxylic groups of ALG and Fc-CHI.

### Characteristics of PEC Hydrogels

Several characteristics of the manufactured PEC were studied. Ninhydrin test, turbidity test, viscosity test, FTIR, swelling ratio, gel content study, and SEM/EDS imaging were performed to evaluate the different properties the hydrogel.

### Degree of Conjugation of Fc in CHI

Ninhydrin assay was used to determine the degree of conjugation. The degree of conjugation of low Fc-CHI, medium Fc-CHI and high Fc-CHI was studied with the reference of pure CHI. The degree of conjugation increases with increase in concentration of Fc in Fc-CHI. The degree of conjugation in low Fc-CHI is 13.37%, medium Fc-CHI is 23.47% and for high Fc-CHI is 42.47%, respectively. This signifies that there is less amount of amino group in the high Fc-CHI. As the degree of polymer conjugation increases, the density of the polymer network also increases. Consequently, as the available free space for drug diffusion decreases, the rate of drug release also decreases. The properties of a (CHI/ALG) material depend on the degree of conjugation between the CHI and ALG. When Fc is added to the CHI, there is an increased degree of conjugation between the Fc-CHI. This results in fewer amino groups available to react with the ALG, leading to a lower overall degree of conjugation in the resulting Fc-CHI/ALG material compared to the pure CHI/ALG material. As a result, the Fc-CHI/ALG material has a higher swelling ratio and higher drug release compared to the pure CHI/ALG material.

### Stoichiometric Ratio

The stoichiometric ratio of polycations and polyanions in a PEC solution is determined by measuring the turbidity of the solution. The turbidity of the solution is expected to increase up to a certain point and then decrease, with the peak point indicating the stoichiometric ratio of the polymers. The increase in turbidity is thought to be due to the phase separation of oppositely charged polyelectrolytes into colloidal particles. The turbidity is also expected to be directly proportional to the polymer concentration, with higher concentrations resulting in higher turbidity. The turbidity curves obtained were different for different PEC concentration.

The stoichiometric point was different for pure CHI/ALG, low Fc-CHI/ALG, medium Fc-CHI/ALG and high Fc-CHI/ALG. Turbidity value of 886, 1102, 1206, and 1344 was obtained in 40% CHI in pure CHI/ALG PEC, 50% Fc-CHI in low Fc-CHI/ALG, 55% Fc-CHI in medium Fc-CHI/ALG and 55% Fc-CHI in high Fc-CHI/ALG. It can be inferred that a higher charge density of the polyanions in a PEC solution shifts the theoretical point of neutralization, or the point at which the positive and negative charges in the solution are balanced, towards a higher ratio of polycations. This means that a larger amount of polycations would be required to neutralize the anionic charge, as the charge density of the anions is higher. The turbidity of a PEC solution decreases after reaching the stoichiometric point, or the point at which the positive and negative charges in the solution are balanced, because excess positive charge can cause destabilization of the PEC and reduce its light scattering affinity. Overall, the stoichiometric ratio of the PEC forms a ionically stable hydrogel, thus the hydrogel doesn't dissolve in the solvent. Also, ionically stable hydrogel helps in controlling the release by preventing the electro-osmotic release and enhancing the electrochemical drug release.

### Mechanical Property

The highest viscosity of a cellulose chain-based hydrogel is achieved at a specific concentration ratio where the strongest intermolecular interactions occur among the cellulose chains, and that this high viscosity leads to a high compressive strength in the hydrogel. Viscosity of combined CHI/ALG rises to 10-fold higher than that of the pure CHI or ALG when mixed in the ratio of 1:1. The viscosity measurement of the stoichiometric proportion of each PEC was performed and concluded that the addition of the Fc in the solution reduces the viscosity. The viscosity decreases due to disruption of the dynamic crosslinking in the hydrogel network. The highest viscosity of 2021.19 cP was obtained for CHI/ALG hydrogel solution, then it successively reduces in each sample according to the amount of Fc content i.e., 1472.53, 1286.27 and 1171.2 for low, medium, and high Fc-Chi/ALG, respectively. The compressive strength of the polymer is affected by the addition of crosslinker. It can be inferred that the compressive strength of pure CHI/ALG PEC is highest and the strength of high Fc-CHI/ALG is lowest. The highest compressive CHI/ALG PEC signifies that this PEC is less affected by the external stimulus. Thus, the degradation of the hydrogel is very difficult. This stiffness can be the reason for retardation in drug delivery. For this reason, the high Fc conjugated hydrogel is effective in the application of drug release.

### FTIR

A FTIR spectrum of a sample is used to identify the types of bonds present in the sample and the functional groups that contain those bonds. The characteristic peak of CHI is between 1649 and 1652 cm⁻¹. Similarly, the peaks for alginate were around 3250 cm⁻¹, 1580 cm⁻¹, and 1398 cm⁻¹. The peak of CHI is obtained at 1638 and 1541 cm⁻¹. Similarly, the peak of ALG is obtained at 1591 and 1417 cm⁻¹. The peak of CHI/ALG PEC is seen at 1587 and 1414 cm⁻¹.

The presence of the Fc in the hydrogel is proved through the FTIR spectra. The functional group of the Fc, organometallic ring around 1500 cm⁻¹. The peak around 540 cm⁻¹ denotes the Fe—C bond and the peak of Fc-CHI is around 1654 cm⁻¹, 1596 cm⁻¹, and 814 cm⁻¹. The peak observed in this experiment is 1655, 1535, and 832 cm⁻¹. The presence of peaks at 1592, 1420, and 826 cm⁻¹ is proof of the formation of PEC between Fc, CHI, and ALG. The absorbance peak of CHI around 1638 cm⁻¹ decreases in Fc-CHI as seen in 1655 cm⁻¹ because of a reduction in primary amine N—H bending. The reduction of peak absorbance in Fc-CHI/ALG than in CHI/ALG is due to the reduction of the degree of ionic interaction between the negatively charged carboxylic ion group of ALG and the positively charged amino group of CHI due to the introduction of Fc. The reduction in the degree of ionic interaction helps in showing the faster response to the electric response, thus the release is faster form the Fc-CHI/ALG PEC. Fc in itself is a redox mediator, so it has the antioxidant property which is required for inhibition of the bacterial growth for wound healing.

### Morphology and Elemental Analysis

Scanning electron microscopy (SEM) is a widely used technique for imaging the surface morphology and structure of materials at high resolution. The information obtained from SEM is also used to understand the physical properties of the material. Stability of a PEC under different environmental factor such as PBS can be determined with SEM as SEM could be used to visualize the network structure of the sponge. The SEM images of the PECs at 100× magnification is taken. When the Fc is added, the structure looks much rougher and flakier. The reduction in crosslinking could leads to a more open, flexible structure for the polymer, which affects its physical properties such as pore size and drug release rate, for this reason the drug release is much faster from Fc-CHI/ALG. Fc-CHI/ALG has larger pore size which enables it to hold a greater quantity of drugs. The network's uneven geometry traps the drugs within its pores and this structure prevents the occurrence of burst release.

Energy dispersive spectroscopy (EDS) is a technique that has application in identifying the elemental composition of a material. EDS works by bombarding the surface of the sample with a beam of high-energy electrons and measuring the energy of the x-rays that are emitted from the surface as a result of the electron interactions with the atoms in the sample. Elemental composition of Fc-CHI is analyzed with EDS. The EDS of pure CHI and Fc-CHI show different results. Chitosan itself does not contain silicon or chlorine; it is possible that impurities from the source material (like, shrimp shells) could be present in the final product. A small hump of Fe at 0.7 keV is seen in Fc-CHI spectra which conforms the presence of iron in Fc-CHI. This small hump of Fe conforms that the Fc-CHI has the presence of Fc in the PEC. As the Fc contains functional group that enhance the solubility or permeability of the drug molecules, leading to more efficient drug release, and the property to inhibit the bacterial growth.

### Weight % of Different Element in Pure CHI and Fc-CHI

| | |
|---|---|
| Pure CHI | Fc-CHI |

| Element | Weight % | Element | Weight % |
|---|---|---|---|
| Carbon | 52.64 | Carbon | 51.15 |
| Nitrogen | 12.37 | Nitrogen | 15.08 |
| Oxygen | 34.99 | Oxygen | 33.69 |
| Iron | 0.00 | Iron | 0.08 |

### Swelling Ratio

The swelling ratio of a PEC can be used to determine the drug release profile of the PEC. The ionized amino groups in the PEC can contribute to the relaxation of the matrix and increase the uptake of the medium by repulsing each other.

The swelling ratio of for CHI/ALG PEC, low Fc-CHI/ALG PEC, medium Fc-CHI/ALG PEC, and high Fc-CHI/ALG PEC were 4965.47%, 5676.88%, 5785.38%, and 5912.66%, respectively. When carboxylic group (—COOH—) is ionized to (—COO⁻—), it increased the internal electrostatic repulsion among the polymer chains which consequently swells the polymeric network. The electrostatic repulsion force decreases as the —COO⁻ combines with H⁺ ions to the form (—COOH—), reducing the electrostatic repulsion force and thus receiving in a shrunken configuration at low pH. The addition of more positively charged cation causes the PEC to be non-stoichiometric, thus there is excessive swelling. The degree of crosslinking can affect the rate uptake of medium for the hydrogel, as the degree of crosslinking is higher the swelling is higher. As the uptake of the drug is enhanced with the high Fc-CHI/ALG PEC, there is effective release of the drug from this PEC to the bacterial environment and can promote the healing of the wound.

### Gel Content

Gel content experiment gives the information for the stability of PEC. The gel contents for pure CHI/ALG, low, medium, and high Fc-CHI/ALG, are 93.82%, 93.99%, 94.01%, and 94.48%, respectively. The gel content of a polymer is related to the amount of free volume or pores present in the material. Polymers with a more dense, rigid structure will tend to have less free volume and therefore a lower gel content. This is because the gel content is a measure of the amount of polymer that is able to move and flow within the material, and a more densely packed structure will have less space for the polymer chains to move around. Conversely, polymers with a more open, flexible structure will tend to have more free volume and a higher gel content. As the overall degree of conjugation of the high Fc-CHI/ALG is lower, the overall gel content percentage of the high Fc-CHI/ALG is higher. This property is also useful because the lower gel content signifies that there is more space for the polymer chains to move, which helps is migration of the drug inside the PEC. An enhanced release can be achieved with the external stimulus.

### Drug Release on the Phantom Skin (Agarose)

The movement of drugs within hydrogels is mainly influenced by diffusion and electrostatic interaction. As the hydrogel containing the drug is exposed to the surrounding environment, the drug can diffuse out of the hydrogel through its pores. Additionally, electrostatic interactions between the hydrogel matrix and the drug can affect its migration. CHI and ALG are polyelectrolytes that contain charged groups, allowing them to interact with oppositely charged molecules such as FITC. FITC is an anionic molecule that can interact with the positively charged CHI component of the hydrogel, slowing down its release and affecting its migration within the hydrogel.

In the case of Fc-CHI/ALG PEC hydrogel, the Fc component serves as a redox-active unit that interacts with an applied electric field, generating a concentration gradient within the hydrogel. This gradient can affect the migration of FITC and other charged species within the hydrogel. When an electric field is applied, the Fc-component undergoes an electrochemical oxidation on the anode side, producing protonated ferrocene (Fc⁺). The protonated ferrocene moieties provide the hydrophilic molecular environment, which increases a wettability on the anode side. The water molecules along with drug migrate towards the anode. Since the CHI component of the hydrogel is conjugated to Fc, it also undergoes an electrochemical reaction, generating a concentration gradient that can affect the migration of FITC and other charged species within the hydrogel. Overall, the Fc component plays a crucial role in generating a concentration gradient within the hydrogel that can affect the migration of FITC and other charged species, which is important for the controlled release of drugs from hydrogels.

Drug Release: Passive Vs. Active

The concentration of drug uptake in a hydrogel is related to the intensity of light collected from the hydrogel through a process called fluorescence. Fluorescence occurs when light is absorbed by the hydrogel and causes the release of energy in the form of light. The intensity of the light emitted from the agarose gel is directly proportional to the concentration of the drug in the agarose gel. Thus, by measuring the intensity of the light emitted from the agarose, it is possible to determine the concentration of the drug within the agarose.

The concentration of FITC absorbed by the hydrogel was found to be 0.02, 0.15, 0.22, and 0.28 mg/ml for pure CHI/ALG, low Fc-CHI/ALG, medium Fc-CHI/ALG, and high Fc-CHI/ALG, respectively. The increasing trend of the drug uptake is directly proportional to the swelling ratio because drug uptake and release is affected by the structural changes due to the degree of crosslinking. The increase in ionic strength causes the decrease in swelling ratio. As the ionic strength of the Fc content hydrogel is lower than that of the pure CHI/ALG. The swelling ratio became lower thus there was more possible area of the ionic drug uptake.

In the intensity profile, the slope of the intensity is increasing successively with the increase amount of Fc in the PEC. The intensity raises with passage of FITC form pure CHI/ALG, low Fc-CHI/ALG, medium Fc-CHI/ALG and high Fc-CHI/ALG PECs without voltage supply was 2962.2 a.u., 11829.6 a.u., 13424.6 a.u. and 13424.6 a.u., respectively.

The release of FITC is less from pure CHI/ALG compared to that of the Fc-CHI/ALG. When the hydrogel is made from stoichiometric ratio of chitosan (CHI) and alginate (ALG) only, the FITC would not migrate towards either the anode or cathode because chitosan and alginate do not have any electroactive groups that can interact with the FITC and drive its migration through electrostatic attraction. Therefore, the FITC would be relatively immobile within the hydrogel, unless it is driven by other physical means such as diffusion or convection.

Addition of Fc in the hydrogel accelerated the change of intensity profile of FITC release. FITC migrate towards the anode (positive electrode) when it is conjugated to a hydrogel containing ferrocene. This is because FITC is a negatively charged molecule and it is being drawn towards the positively charged electrode by electrostatic attraction. The ferrocene in the hydrogel is also contributing to the migration of FITC by electrostatic attraction.

Along with the application of voltage the release of FITC is highly increased. The release reached its saturation at 4 min from all hydrogels. The saturation is predicted with the plateau at 14000 a.u., which indicates that the intensity has reached the maximum limit for given material. The time to reach saturation from low Fc-CHI/ALG, medium Fc-CHI/ALG and high Fc-CHI/ALG is 2.5 min, and form the pure CHI/ALG is 4 min. The release trend with increasing amount of Fc is similar. It is observed that the intensity increases sharply while FITC is being released form pure CHI/ALG as compared to that of the passive release. This can be explained because when the hydrogel is made from chitosan (CHI) and alginate (ALG) only and an electric field is supplied, the FITC would migrate towards the anode (positive electrode) under the influence of the electric field. The FITC is a negatively charged molecule and it will be drawn towards the positively charged electrode by the electric field. Although chitosan and alginate do not have any electroactive groups that can interact with the FITC, the electric field will provide the necessary force to drive the FITC migration through electrostatic attraction.

When incubated in 1 mg/ml FITC-Dextran, the concentration of drug uptake by pure CHI/ALG, low Fc-CHI/ALG, medium Fc-CHI/ALG and high Fc-CHI/ALG was 0.052, 0.103, 0.117 and 0.158 mg/ml, respectively. The comparative release of FITC-Dextran is slower than that of the pure FITC. FITC Dextran has the molecular weight of 66 kDa which is the main reason behind the slow passage from the PEC to the agarose gel because the higher the molecular weight slower the drug migrates through the hydrogel.

As like that of the FITC release FITC-Dextran has also been released from different PEC along with the different voltage application. From the intensity change observance, the release of FITC-Dextran from no electric current supplied were 2053.2 a.u., 5176.2 a.u., 8826.8 a.u. and 11135.8 a.u. from pure CHI/ALG, low Fc-CHI/ALG, medium Fc-CHI/ALG and high Fc-Chi/ALG, respectively.

It is seen that the release of FITC-Dextran from Fc-CHI/ALG is higher than that of pure Chi/ALG PEC. This is because the release is favored with only diffusion. As the pore size of the Fc-CHI/ALG PEC is higher than that of pure CHI/ALG there is more diffusion channel for passage of more drug.

The release of incorporated FITC-Dextran with 2V, 1.5 A electric stimulus from pure CHI/ALG, low Fc-CHI/ALG, medium Fc-CHI/ALG and high Fc-CHI/ALG was found to be 4779 a.u., 7021.6 a.u., 11848.6 a.u. and 12276.4 a.u., respectively. It is observed that the FITC-Dextran started to migrate towards the anode much faster than as compared to that of the not applied with electric stimulus. The FITC-Dextran is a negatively charged molecule because of the presence of isothiocyanate group in FITC and the dextran is also negatively charged because it is a polysaccharide. From the study performed by Jenson and his team it was found that there is direct relation of electrostatic force driving the negatively charged drug sulfosalicylic acid towards oppositely charged electrode, thus diffusion coefficient is higher even for the material having same crosslinking ratio or mesh size. Due to same effect when an electric field is applied, the negatively charged FITC-Dextran will be drawn towards the positively charged anode. Fc can act as a reducing agent, which can break the cross-links in the hydrogel and increase its degradation, leading to a faster release of the FITC-Dextran. Additionally, Fc can alter the surface charge and hydrophilicity of the hydrogel, which can facilitate the diffusion of FITC-dextran and enhance its release.

The amount of FITC-BSA absorbed was 0.248 and 0.401 mg/ml for pure CHI/ALG and high-Fc-CHI/ALG, respectively. The release of predicted FITC-BSA form the rise of intensity pure CHI/ALG and Fc-CHI/ALG is 335.4 a.u. and 1433.8 a.u., respectively. When a hydrogel is made from only CHI and alginate ALG and fluorescein isothiocyanate-bovine serum albumin (FITC-BSA) is released, it is observed that, very less amount of FITC-BSA is released as compared to FITC this is due to the molecular size of FITC-BSA is larger than that of pure FITC. As similar to that of FITC, FITC-BSA also travels towards the anode. This is due to the fact that chitosan is positively charged, and alginate is negatively charged, creating an electric potential gradient in the gel that drives the negatively charged FITC-BSA towards the positively charged anode. The study performed by Ju Young found that the negatively charged FITC-BSA migrated towards anode due to electrophoresis. They studied the diffusivity of the FITC-BSA on 2 wt % agarose gel. Their comparative study of diffusivity of Rhodamine and FITC-BSA showed that FITC-BSA is negatively charged since its migration is towards anode while the migration of Rhodamine towards cathode proves that this is positively charged. The addition of ferrocene to a hydrogel can increase the release of an encapsulated molecule, such as FITC-BSA, due to the redox (reduction-oxidation) properties of ferrocene. Ferrocene can transfer electrons from one molecule to another and can therefore cause changes in the local pH and redox potential of the hydrogel. These changes can in turn lead to changes in the structure and properties of the hydrogel, such as increased porosity or altered charge distribution, which can enhance the release of the encapsulated molecule. Additionally, the presence of ferrocene in the hydrogel can also influence the diffusion of the encapsulated molecule through the hydrogel, contributing to the increased release rate.

Along with the application of 1.5 V electric stimulus, we observed a little change in release of FITC-BSA. The intensity of Agarose gel obtained from pure CHI/ALG and Fc-CHI/ALG was 1198.6 a.u. and 1922.8 a.u., respectively. When an electric field is supplied to a chitosan-alginate hydrogel, the release of an encapsulated molecule can be faster. This is due to the electrical stimulation of the hydrogel, which creates an electro-osmotic flow that helps to drive the release of the encapsulated molecule. The electric field can also cause the hydrogel to deform and change its structure, further increasing the release rate. Additionally, the application of an electric field can also enhance the diffusion of the. Increasing the amount of ferrocene in the hydrogel will increase the overall charge distribution within the hydrogel, which can lead to a stronger electro-osmotic flow and an increased release of FITC-BSA in response to an electric field. The amount of ferrocene present in the hydrogel is related to the release of FITC-BSA in response to an electric field.

### Dynamic Release

The cyclic voltage was applied in both directions, i.e., from cathode-anode-agar and from anode-cathode-agar. Since the migration of the drug in the incorporation of the Fc⁻ is driven towards the anode, the release through the cathode-anode-agar is faster than in the opposite direction. The release of FITC is dependent on the presence of the Fc. The intensity profile with a 1.5V electric field shows that the drug migrates towards the anode with Fc-CHI/ALG PECs, and from pure CHI/ALG, the migration of drugs is slower. After 10 minutes of release, the intensity reaches 4600 and 11355 μm from pure CHI/ALG and Fc-CHI/ALG hydrogels, respectively. The release of FITC in the opposite direction, i.e., anode-cathode-agar is much slower. At the end of 10 minutes, the release was 3971 a.u., 6991 a.u., and 8773 a.u. from pure CHI/ALG, low Fc-CHI/ALG, and medium Fc-CHI/ALG hydrogel, respectively. The release is accelerated while the voltage is ON (cathode-anode-agar), and the release is steady comparatively while the voltage is OFF. The intensity at the end of 5 minutes of 3V application of the release was 8610 a.u. and 11787 a.u. from pure CHI/ALG and all Fc-CHI/ALG, respectively. The release from the opposite direction, i.e., anode-cathode-agar, is unclear as the intensity reaches 595 a.u. from pure CHI/ALG, and it is only 8344 a.u. from Fc-CHI/ALG at the end of 5 minutes. The migration of drugs towards the anode was faster, resulting in a higher release rate through cathode-anode-agar. The presence of Fc-CHI/ALG PECs also influenced the release rate of FITC, and the application of an electric field accelerated the release.

The release of FITC-Dextran in a dynamic environment is similar to that in a static environment. The application of voltage in a cyclic manner shows the slight change in slope while transitioning from the off state to the on state. After 15 minutes of cyclic release of FITC-Dextran with 1.5V, the intensity was found to be 5545 a.u., 8150 a.u., 9045 a.u. and 11076 a.u. from pure CHI/ALG, low Fc-CHI/ALG, medium Fc-CHI/ALG, and high Fc-CHI/ALG respectively. Following the same process, after 10 minutes of release of FITC-Dextran with 2 V, the intensity was found to be 5815 a.u., 6830 a.u., 6830 a.u. and 12086 a.u. from pure CHI/ALG, low Fc-CHI/ALG, medium Fc-CHI/ALG and high Fc-CHI/ALG respectively. The results indicate that the release of FITC is highly controllable, with clear and distinct slopes in the on and off conditions, however the release of FITC-Dextran shows only a slight change in slope. The release time of FITC is faster with a 2V voltage compared to 1.5V, as it reaches saturation in half the time. Similarly, FITC-Dextran shows faster release after 10 minutes with a 2V voltage compared to 15 minutes with a 1.5V voltage. The results also suggest that the presence of Fc in the hydrogel makes it more electrochemically active PEC (E-PEC), as the release was greatly affected by the application of voltage in the high Fc-CHI/ALG.

It can be concluded that E-PEC hydrogel, specifically high Fc-CHI/ALG, is the best choice for use in wound healing purposes due to its controllable release of drugs and faster release time with the application of voltage. Its electrochemical activity and ability to enhance the release of drugs make it a promising candidate for wound healing applications.

### In-Vitro Biofilm Assay: Antimicrobial Effect

The purpose of this experiment was to observe effectiveness of drug release on bacterial biofilms through the hydrogel. The selected hydrogel for this observation was E-PEC because of its enhanced properties like larger pore with irregular polymeric geometry, increased swelling ratio and electro-responsivity. This hydrogel is best for the release of the FITC and FITC release. The elimination of both gram-positive and gram-negative bacteria was observed using CIP antibiotics. It was noted that S. aureus is susceptible to certain types of antibiotics due to its thick cell wall as a gram-positive bacterium, while P. aeruginosa, a gram-negative bacterium with a thinner cell wall and outer membrane, is typically resistant to antibiotics.

The effectiveness of the release of CIP over S. aureus was compared with the release of the control, PBS, in an experiment. The hydrogel E-PEC was loaded with either PBS or CIP, and the release was performed in three ways: CIP/E-PEC, E-PEC loaded with PBS, and a control group treated with PBS only. The CIP/E-PEC group was also subjected to an electric voltage of 2 V.

CFU (colony-forming unit) counts were taken at specific time points. The control group showed a slight increase in CFU count overtime, from 144×10⁷/ml to 165×10⁷/ml. The electric device was designed to show the effectiveness of the encapsulated hydrogel under electric stimulus, as demonstrated by the result of the E-PEC+ stimulus, where the CFU count increased from 144 to 150×10⁷/ml. The E-PEC group showed a slight increase in CFU count in the third hour, but by the end of the experiment, the CFU count was the same, indicating that the E-PEC does not inhibit but prevents the bacterial growth. The release of CIP without stimulus (CIP/E-PEC) resulted in a reduction in CFU count to 90×10⁷/ml, while the release of CIP with electric stimulus was highly effective, reducing the CFU count to 50×10⁷/ml. In summary, the control group showed slight increase in CFU counts over time. The release of CIP with electric stimulus was highly effective in reducing CFU counts, while the hydrogel alone had no effect on the biofilm.

The hydrogel E-PEC was also used to test its efficacy in reducing growth of gram-negative bacterial biofilms. The results showed that the combination of CIP and electric stimulus treatment led to a decrease in CFU count from 145×10⁷/ml to 78×10⁷/ml over six hours. In contrast, the CFU count of the biofilm treated with CIP alone was 105×10⁷/ml after six hours. The biofilm treated with E-PEC alone had a higher CFU count of 162×10⁷/ml after six hours, indicating that it was ineffective in killing the bacteria. The control group treated with PBS showed an increase in CFU count from 144 to 190×10⁷/ml after six hours.

The results indicate that the combination of CIP and electric stimulus treatment using the E-PEC hydrogel was effective in reducing bacterial growth in biofilms. The use of CIP alone showed some efficacy, but it was not as effective as the combination treatment. Treatment with the E-PEC hydrogel alone was found to be ineffective in killing the bacteria. The E-PEC hydrogel used in the experiment have acted as a controlled-release system. The electric stimulus has enhanced the release of the antibiotic from the hydrogel, allowing it to reach the bacterial cells more effectively within the biofilm. Controlled release of antibiotics from hydrogels was particularly effective. Biofilms are complex structures formed by bacteria that can provide a protective environment for the microorganisms, making them more resistant to traditional antibiotic treatments. The controlled release of antibiotics through the different voltage supply from hydrogels can allow for a more gradual and prolonged exposure of the bacteria to the drug, which can increase the likelihood of eradicating the biofilm.

Additionally, controlled release from hydrogels can also help reduce the frequency of dosing, which can be particularly beneficial for patients who require long-term antibiotic therapy. By reducing the need for frequent dosing, controlled-release hydrogels can improve patient compliance and reduce the risk of side effects associated with high-dose antibiotic therapy. In summary, controlled release of antibiotics from hydrogels can improve the effectiveness of antibiotics against bacterial biofilms by providing a continuous and controlled release of the drug over shorter or extended period of time. This controlled exposure can increase the likelihood of eradicating biofilm and reduce the frequency of dosing, which can improve patient compliance and reduce the risk of side effects.

The combination of the Fc, CHI and ALG hydrogel can be used in multiple stages of wound healing like, hemostasis due to adhesion property provided by ALG, inflammatory due to the anti-bacterial property provided by positive charges of CHI. Fc also helps in reducing the inflammation through the redox property. The inhibition of the bacterial colony observed in the in-vitro experiment helps to clarify that this E-PEC hydrogel system can be used in the inflammatory stage of the wound healing. As well as the E-PEC also consists of the ALG, so it can be used for the hemostasis stage of wound healing.

### CONCLUSIONS AND RECOMMENDATIONS

PEC hydrogels are made from CHI/ALG, and E-PEC hydrogels are made with addition of varying amounts of Fc. First the CHI was conjugated with Fc to produce Fc-CHI and was lately crosslinked with ALG.

CHI/ALG was used as a control and the properties of Fc-CHI/ALG were studied in reference to it.

The results of UV testing on wash solutions suggested that there was no free Fc present in Fc-CHI flakes/granules.

The degree of conjugation was measured by Ninhydrin assay, which indicated an increasing trend of free amines from low, medium, and high Fc-CHI based on light absorption. The percentage of free amines in low Fc-CHI, medium Fc-CHI and high Fc-CHI is 13.37%, 23.47% and 42.47%, respectively.

The turbidity test revealed that adding Fc changed the stoichiometric proportion of polycation and polyanion. The stoichiometric proportion of the pure CHI/ALG, low Fc-CHI/ALG, medium Fc-CHI/ALG and high Fc-CHI/ALG is formed at 40% CHI, 50% Fc-CHI, 55% Fc-CHI and 55% Fc-CHI, respectively.

The peak shifted towards higher proportions of Fc-CHI, indicating Fc-conjugation with CHI and a lower number of free amines, requiring reaction with ALG.

Viscosity testing of the stoichiometric proportion demonstrated that the mechanical strength of PEC decreased with increasing amounts of Fc. The viscosity of pure CHI/ALG, low Fc-CHI/ALH, medium Fc-CHI/ALG and high Fc-CHI/ALG was 2021.9 cP, 1472.52 cP, 1286.27 cP and 1171.2 cP, respectively.

Gel content 94.48%, 94.01%, 93.99% and 93.82% was obtained from pure CHI/ALG, low Fc-CHI/ALG, medium Fc-CHI/ALG and high Fc-CHI/ALG, respectively. Similarly, the swelling ratio of 4965.47%, 5676.88%, 5785.38 and 5912.66%, respectively.

The increasing concentration of the Fc in the PEC increased the pore size of PEC and changed the surface morphology, which can be used to hold larger amount of drugs within its pore.

Gel content and swelling ratio test results increased with an increase in Fc, as evidenced by SEM images showing larger pore size in PEC with more Fc.

The static release of FITC from PEC changed the concentration in agarose gel, which increases the intensity of the agarose gel.

The intensity changes after 4 minutes in agarose gel obtained with 0V electric stimulus from pure CHI/ALG, low Fc-CHI/ALG, medium Fc-CHI/ALG and high Fc-CHI/ALG was 2962.2, 11829.6, 13424.8 and 13424.8 a.u., respectively. Similarly, with application of 2V the intensity of the all Fc-CHI/ALG was 14000 at 3 minutes and after 4 minutes the intensity of pure CHI/ALG was 14000 form pure CHI/ALG.

FITC-Dextran was released slowly as compared to that of the FITC. The release of FITC-Dextran with 0V electric stimulus increased the intensity to 2053.2, 5176.2, 8826.6 and 12276.4 a.u., respectively, whereas with 2V the intensity was 4779, 7021.6, 11848.6 and 12276.4 a.u., respectively.

FITC-BSA release with 0V was 335.4 and 1433.8, respectively, with 2V electric stimulus the release of the FITC-BSA increased the intensity in the agarose gel to 1198.6 and 1922.8, respectively.

The release was highest with high Fc-CHI/ALG in the presence of electric current and lowest with CHI/ALG in the off state of voltage supply, leading to an increase in E-PEC.

Drug release kinetics were performed on Agarose with voltage on and off from each PEC, ranging from pure CHI/ALG to high Fc-CHI/ALG.

The release of drug with cyclic voltage application showed a stair like release pattern, this signifies the prompt response of the hydrogel towards electric stimulus in releasing drugs.

The faster responsiveness of high Fc-CHI/ALG signifies that that the Fc-CHI/ALG is electrically active polyelectrolyte complex (E-PEC).

The release of the CIP from the E-PEC with electric stimulus reduced the CFU×10⁷/ml of S. aureus was 144 to 41. Similarly, the release of CIP from the E-PEC with electric stimulus reduced the CFU×10⁷/ml of P. aeruginosa from 145 to 71.

Future work can be focused on use of the different antibiotics or mixed antibiotics of methicillin, vancomycin, ampicillin, tetracycline and cefoxitin, for the effective inhibition antibiotics on S. aureus, P. aeruginosa, and Methicillin-resistant S. aureus (MRSA) biofilms.

Antimicrobial effect of the E-PEC hydrogel on biofilm demonstrated an enhanced effect on antibiotic release, as evidenced by the reduced CFU values on the treated biofilms. This eradication of the biofilm makes the E-PEC hydrogel suitable for inflammatory stage of the wound healing while the presence of ALG would be effective for hemostasis stage of the wound healing.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A ferrocene-containing polyelectrolyte complex.

2. The ferrocene-containing polyelectrolyte complex of claim 1, wherein the ferrocene-containing polyelectrolyte complex comprises ferrocene-conjugated chitosan as a polycation and alginate as a polyanion.

3. The ferrocene-containing polyelectrolyte complex of claim 1, formed by a process comprising:
bonding ferrocene to chitosan to form ferrocene-conjugated chitosan; and
reacting the ferrocene-conjugated chitosan with alginate.

4. A composition comprising:
a ferrocene-containing polyelectrolyte complex.

5. The composition of claim 4, further comprising:
an active ingredient loaded in the ferrocene-containing polyelectrolyte complex.

6. The composition of claim 4, wherein ferrocene is bonded to a polycation of the polyelectrolyte complex.

7. The composition of claim 6, wherein the polycation is chitosan.

8. The composition of claim 4, wherein a polyanion of the ferrocene-containing polyelectrolyte complex is alginate.

9. The composition of claim 6, wherein the polycation is chitosan; and wherein a polyanion of the ferrocene-containing polyelectrolyte complex is alginate.

10. The composition of claim 9, wherein a weight ratio of the ferrocene-conjugated chitosan to alginate is in a range of from greater than 0:1 to less than 10:0.

11. The composition of claim 9, wherein a weight ratio of the ferrocene-conjugated chitosan to alginate is in a range of about 4:6 to about 6:4.

12. The composition of claim 9, wherein a weight ratio of the ferrocene-conjugated chitosan to alginate is about 5.5:4.5.

13. The composition of claim 12, wherein a weight ratio of ferrocene to chitosan in the ferrocene-conjugated chitosan is in a range of about 0.013 to 0.052.

14. A wound dressing comprising:
a cathode;
an anode; and
a composition located between the cathode and the anode, the composition comprising:
a ferrocene-containing polyelectrolyte complex; and
an active ingredient.

15. The wound dressing of claim 14, further comprising:
a polydimethylsiloxane chamber;
wherein the composition is located within the polydimethylsiloxane chamber.

16. The wound dressing of claim 14, wherein the cathode and the anode comprise laser-induced graphene.

17. The wound dressing of claim 14, further comprising:
an adhesive layer on a side of the anode opposite the composition.

18. The wound dressing of claim 14, wherein the ferrocene-containing polyelectrolyte complex comprises ferrocene-conjugated chitosan as a polycation and alginate as a polyanion.

19. The wound dressing of claim 18, wherein a weight ratio of the ferrocene-conjugated chitosan to alginate is in a range of about 1:5 to about 1:1.

20. The wound dressing of claim 18, wherein a degree of ferrocene conjugation of the ferrocene-conjugated chitosan is in a range of about 55% to about 84%.
